(19) United States
(12) Patent Application Publication
(10) Pub. No.: US 2018/0361032 A1
(43) Pub. Date: Dec. 20, 2018

(54) IMPLANTABLE ELECTROSPUN PATCHES FOR SITE-DIRECTED DRUG DELIVERY

(71) Applicant: The Methodist Hospital, Houston, TX (US)

(72) Inventors: Ennio Tasciotti, Houston, TX (US); Francesca Taraballi, Houston, TX (US); Silvia Minardi, Houston, TX (US)

(21) Appl. No.: 16/109,198

(22) Filed: Aug. 22, 2018

Related U.S. Application Data
(63) Continuation of application No. PCT/US2017/000017, filed on Feb. 22, 2017.
(60) Provisional application No. 62/298,407, filed on Feb. 22, 2016.

(51) Int. Cl. A61L 31/16; A61L 27/54; A61L 31/14; A61L 27/58

(52) U.S. Cl. CPC A61L 31/16; A61L 27/54; A61L 17/06; A61L 27/58; A61L 31/148

(57) ABSTRACT

Disclosed are biocompatible, biodegradable, implantable devices for the controlled release of bioactive molecules. In particular embodiments, nanotechnology-based tunable implants are disclosed for 1) localized delivery of analgesics to treat postoperative pain; 2) sustained delivery of growth factors to promote vascularization; and 3) directing tissue regeneration, including the self-direction of autologous stem cells for organ remodeling.

### CROSS-REFERENCE TO RELATED APPLICATIONS

The present application claims priority to PCT International Patent Application No. PCT/US2017/000017, filed Feb. 22, 2017 (nationalized; Atty. Dkt. No. 37182.192WO01); which claims priority to U.S. Provisional Patent Application No. 62/298,407, filed Feb. 22, 2016 (expired; Atty. Dkt. No. 37182.192PV01); the contents of each of which is specifically incorporated herein in its entirety by express reference thereto.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### BACKGROUND OF THE INVENTION

### Field of the Invention

The present invention relates generally to the fields of medicine and surgery, and in particular, to implantable drug delivery systems. Disclosed are biocompatible, biodegradable, implantable devices for the controlled release of bioactive molecules. In particular embodiments, nanotechnology-based tunable implants are disclosed for 1) localized delivery of analgesics to treat postoperative pain; 2) sustained delivery of growth factors to promote vascularization; and 3) directing tissue regeneration, including the self-direction of autologous stem cells for organ remodeling.

### Description of Related Art

Drug Delivery Systems

Numerous devices have previously been developed for the delivery of drugs and other medicinal compounds. Transdermal devices are technically capable of slowly administering drugs at a constant rate over an extended period of time, however; they often fail to consistently deliver all of the drug beneath the stratum corneum layer of the skin so that it can be absorbed into the body. When this occurs, all or a portion of the drug is delivered only onto the top of the skin or into the stratum corneum layer, where the drug cannot be absorbed into the body of the patient.

Transdermal devices are poorly suited for the localized treatment of acute pain, and are wholly unsuitable for treating postoperative incisional pain, or for delivering biological agents such as analgesics to deep tissue sites, or to organs within the body.

Existing implantable drug delivery devices also are unsuitable for these applications for a variety of reasons. In particular, no biocompatible patches have been developed to date that are capable of growing with the patient, or for remodeling the structure or matching the structure of existing tissue.

Therefore, what is lacking in the art is an implantable, drug delivery device that is biocompatible, biodegradable, and has an improved ability to consistently and effectively deliver one or more drugs, such as analgesics, in a localized, targeted, and controlled-release fashion to treat incisional and surgical pain. Furthermore, what is also lacking in the art is a cellularized biopatch that is capable of directing tissue regeneration while growing with the patient, to support native organ structure and restore organ function.

### BRIEF SUMMARY OF THE INVENTION

The present invention overcomes these and other limitations inherent in the prior art by providing biomimetic, bioactive, biocompatible, and biodegradable drug delivery devices that can be implanted to provide a controlled-release of one or more drugs, growth factors, and/or bioactive molecules, to a surgical or deep tissue site, including, for example, the delivery of one or more analgesics to treat incisional pain, and to improve the post-surgical outcome. Also provided is a membrane patch useful in repairing cardiac tissue in situ.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to demonstrate certain aspects of the disclosure. For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The invention may be better understood by reference to the following description taken in conjunction with the accompanying drawings, in which like reference numerals identify like elements, and in which:

FIG. 1A and FIG. 1B show an exemplary analgesic patch for drug delivery in accordance with one aspect of the present invention. FIG. 1A shows the elecrospun layer: nanostructured membrane, porous. The inset (FIG. 1B) shows a magnification of the membrane topography that highlights the nanostructure. All the layers can be further modified with nanoparticles for the release of bioactive molecules;

FIG. 2A, FIG. 2B, and FIG. 2C show an exemplary analgesic patch for drug delivery in accordance with one aspect of the present invention. Elecrospun layer (FIG. 2A): nanostructured membrane, porous plus a compact insulator layer of collagen; FIG. 2B shows a magnification of the membrane topography that highlight the nanostructure; and FIG. 2C shows a magnification of the membrane topography that highlight the insulator compact layer. Each of the layers can be further modified with one or more populations of distinct nanoparticles adapated and configured to contain and release one or more bioactive molecules;

FIG. 3A, FIG. 3B, FIG. 3C, and FIG. 3D show the assembly of a double-layer implant (FIG. 3A) that allows for the directional release of the loaded drug from the porous layer (FIG. 3C). Shown is the release of the drug (red dots) from the nanofibers (FIG. 3B). Each of the layers can be further modified with one or more populations of distinct nanoparticles (e.g., inset FIG. 3D) adapted and configured to contain and release one or more bioactive molecules;

FIG. 4A and FIG. 4B show an exemplary double-layer membrane in accordance with one aspect of the present disclosure. The double-layer membrane allows for the directional release of the loaded drug from the porous layer (FIG. 4A) while the collagen shield blocks the release. In this example, a nerve or bone wrapping (FIG. 4B) can be seen in which it is necessary for the release of growth factor/drug only in one direction confining the release only inside the membrane. Each of the layers can be further modified with one or more populations of distinct nanoparticles adapated and configured to contain and release one or more bioactive molecules;

FIG. 5A, FIG. 5B, FIG. 5C, FIG. 5D, and FIG. 5E show various aspects of the disclosure, including a schematic of the electrospin device views (FIG. 5A), while FIG. 5C, and FIG. 5B respectively show an overall view, and an SEM view, respectively, of a typical BupiPatch in accordance with one aspect of the present disclosure FIG. 5D is a plot of released bupivacaine over time, which illustrates “tuning” of the polymer composition and the ratio of its components. FIG. 5E is a plot of released bupivacaine over time illustrating tuning of the bupivacaine (BV):polymer ratio;

FIG. 6 shows a multi-scale approach to nanotechnologies for drug delivery;

FIG. 7A, FIG. 7B, FIG. 7C, and FIG. 7D show an incisional pain rodent model for assessing analgesic-releasing patches in accordance with one aspect of the present disclosure;

FIG. 8 illustrates the chemical structure of the local anesthetic, lidocaine;

FIG. 9A, FIG. 9B, and FIG. 9C show blocking of the ion-dependent channels (FIG. 9A). At the resting potential, voltage-gated Na⁺ channels are closed (FIG. 9B); when the membrane is depolarized, conformational changes open the voltage-gated channels (FIG. 9C);

FIG. 10 shows exemplary tissue damage in accordance with one aspect of the present disclosure;

FIG. 11 shows an exemplary experimental design in accordance with one aspect of the present disclosure;

FIG. 12A, FIG. 12B, FIG. 12C, FIG. 12D, FIG. 12E, and FIG. 12F show the expression of genes related to pain in sciatic nerve tissue: Scn10a (FIG. 12A); Scn9a (FIG. 12B); Scn3a; PTGES (FIG. 12D); PDE4D (FIG. 12E); and S100A4 (FIG. 12F); respectively;

FIG. 13A, FIG. 13B, FIG. 13C, FIG. 13D, and FIG. 13E show the expression of genes related to inflammation in sciatic nerve tissue: TNF-α (FIG. 13A); IL-1β (FIG. 13B); IL-6 (FIG. 13C); PTGS2 (COX-2) (FIG. 13D); and NOS2 (FIG. 13E); respectively;

FIG. 14A, FIG. 14B, FIG. 14C, FIG. 14D, and FIG. 14E show the expression of genes related to inflammation in surrounding muscle tissue: COX-2 expression (FIG. 14A); IL-6 expression (FIG. 14B); TNF-α expression (FIG. 14C); IL-1β expression (FIG. 14D); and iNOS expression (FIG. 14E);

FIG. 15A, FIG. 15B, FIG. 15C, FIG. 15D, and FIG. 15E show exemplary sciatic nerve (FIG. 15A) histology in Bupipatch (FIG. 15B and FIG. 15C) vs. membrane control (FIG. 15D and FIG. 15E) in accordance with one aspect of the present disclosure;

FIG. 16 shows exemplary clinical benefits of regenerative medicine by combining bioactive molecules, extracellular matrix components, and cells to form tissue in accordance with one aspect of the present disclosure;

FIG. 17 shows that individual delivery of VEGF and PDGF can direct the formation of a mature vasculature in accordance with one aspect of the present disclosure;

FIG. 18 shows that the dual delivery of VEGF and PDGF-BB, each with distinct kinetics, from an electrospun BioPatch functionalized with polymeric drug delivery systems results in the rapid formation of a mature vascular network in accordance with one aspect of the present disclosure;

FIG. 19 shows a schematic for giving cells the right instructions in a regenerative medicine scenario in accordance with one aspect of the present disclosure;

FIG. 20A, FIG. 20B, FIG. 20C, FIG. 20D, FIG. 20E, and FIG. 20F show the encapsulation of porous silicon MSV in PLGA microspheres provides a further level of control over the relase; shown are scanning electron micrographs of MSV particles in accordance with one aspect of the present disclosure; FIG. 20G, FIG. 20H, FIG. 20I, and FIG. 20J show the MSV porous core, the first emulsion, the second emulsion, and the final PLGA-MSV composite, respectively, in accordance with one aspect of the present disclosure; FIG. 20K, FIG. 20L, FIG. 20M, FIG. 20N, FIG. 200, FIG. 20P, FIG. 20Q, FIG. 20R, and FIG. 20S are optical microscopy images recorded in accordance with one aspect of the present disclosure; and FIG. 20T and FIG. 20U are plots of particle diameters as a function of concentration (FIG. 20T) and MSV content (FIG. 20U) in accordance with one aspect of the present disclosure;

FIG. 21 is a plot of percentage release of particles as a function of time for various compositions as shown, in accordance with one aspect of the present invention; PLGA-MSV allowed for the fine controlled release of proteins both in vitro and in vivo;

FIG. 22 shows a method for recreating a proper home for cells to grow in an exemplary regenerative medicine scenario in accordance with one aspect of the present disclosure;

FIG. 23A, FIG. 23B, FIG. 23C, FIG. 23D, FIG. 23E, FIG. 23F, and FIG. 23G show biomimetic material intended to interface with biological systems to treat, remodel, or replace a damaged tissue, in accordance with one aspect of the present disclosure;

FIG. 24A, FIG. 24B, FIG. 24C, and FIG. 24D illustrate parameters of exemplary compositions in accordance with the present invention;; EDC/NHS Crosslinking Surface Shrinking is shown in FIG. 24A; Diameter Frequency is shown in FIG. 24B; Post-vs. Pre-Crosslinking is demonstrated in FIG. 24C; and the Degree of particle swelling is shown in FIG. 24D;

FIG. 25A, FIG. 25B, FIG. 25C, FIG. 25D, FIG. 25E, FIG. 25F, FIG. 25G, and FIG. 25H show that by integrating PLGA-MSV in the collagen mats (camouflage) it is possible to ensure their spatial confinement and the preservation of their payload's release kinestics, which results in an efficient GFs and release of molecules;

FIG. 26 shows a regenerative medicine scenario that permits human stem cell proliferation in accordance with one aspect of the present disclosure;

FIG. 27A and FIG. 27B show hMSCs have the ability to differentiate into multiple lineages such as chondrocytes, osteocytes, and adipocytes; these pluripotent hMSCs can effectively suppress immune responses, and are thus considered to be immunosuppressive;

FIG. 28A, FIG. 28B, FIG. 28C, FIG. 28D, FIG. 28E, and FIG. 28F show exemplary stemness marker genes (surface antigens) present in 3D cultures that performed better when the stemness of hMSCs were maintained over time;

FIG. 29 shows the testing of a material's properties. Human MSC were able to proliferate on the patch and maintain their unique features. Human MSCs did not express inflammatory markers, demonstrating that the patch did not elicit any immune response;

FIG. 30A, FIG. 30B, FIG. 30C, FIG. 30D, FIG. 30E, FIG. 30F, and FIG. 30G show PLGA mesoporous silicon microspheres for the in vivo controlled temporospatial delivery of proteins;

FIG. 31A, FIG. 31B, FIG. 31C, FIG. 31D, FIG. 31E, and FIG. 31F show enhancing vascularization through the controlled relase of PDGF-BB; the results of PLGA-MSV/PDGF-BB are presented compared to control, and the mean fluorescence intensity of samples with CD31 (FIG. 31C) and α-SMA (FIG. 31F) were measured;

FIG. 32A, FIG. 32B, FIG. 32C, FIG. 32D, FIG. 32E, FIG. 32F, FIG. 32G, and FIG. 32H show the controlled release of PDGF-BB by PLGS-MSV particles over 14 days guided what appeared to be a mature netword of vessels;

FIG. 33A, FIG. 33B, FIG. 33C, FIG. 33D, FIG. 33E, FIG. 33F, FIG. 33G, FIG. 33H, and FIG. 33I show a murine model for implantation of an exemplary patch, and measurement of the relative expansion of Vegfa (FIG. 33D), Vegfr2 (FIG. 33E), Vwf (FIG. 33F); and Col3a1 (FIG. 33G);

FIG. 34A, FIG. 34B, FIG. 34C, FIG. 34D, FIG. 34E, FIG. 34F, FIG. 34G, and FIG. 34H show results of a study that monitored the presence of PDGF-BB/VEGF vs. controls at 7, 14, and 21 days;

FIG. 35 illustrates various congenital heart defects, which can affect nearly 1% of live births in the United States (nearly 40,000 births per year); Septal wall defect is the most common, with 25% of those requiring surgical repair;

FIG. 36 shows an exemplary cellularized CardioPatch capable of self-directing autologous stem cells into a cardiac linease, and which grows with the patient and is biodegradable supports not only the native heart structure, but also restores function;

FIG. 37A and FIG. 37B illustrate cell proliferation over 21 days for osteo or chonro cells, as compared to no treatment control;

FIG. 38A, FIG. 38B, FIG. 38C, FIG. 38D, FIG. 38E, FIG. 38F, FIG. 38G, FIG. 38H, FIG. 38I, and FIG. 38J show stemness marker and the degree of relative expansion of SPARC (FIG. 38E) and SPP1 (FIG. 38F) over a 21-day treatment window for an exemplary BioPatch in accordance with one aspect of the present disclosure;

FIG. 39A, FIG. 39B, FIG. 39C, FIG. 39D, FIG. 39E, FIG. 39F, FIG. 39G, and FIG. 39H show stemness marker and the degree of relative expansion of COL1A1 (FIG. 39E) and ACAN (FIG. 39F) over a 21-day treatment window;

FIG. 40A, FIG. 40B, FIG. 40C, FIG. 40D, FIG. 40E, and FIG. 40F show microscopic analyses of cells during a time-course assay of 72 hrs to 14 days;

FIG. 41 shows a method for forming tissues from a combination of biomaterials and cells in an exemplary regenerative medicine scenario in accordance with one aspect of the present disclosure;

FIG. 42A and FIG. 42B show the respective structures of the exemplary glycogen synthase kinase 3 inhibitors, IWP-2 and CHIR-99021HCL; and

FIG. 43 shows an exemplary isolation method for stem cells and the production of a beating CardioPatch for surgical reconstruction of a normal heart structure. Temporal modulation of WNT signaling is essential and sufficient for efficient cardiac induction in human pluripotent stem cell (hPSC). Sequential treatment of hPSC with glycogen synthase kinase 3 (GSK-3) inhibitors followed by chemical inhibitors of WNT signaling produce pure functional human cardiomyocytes from hPSC.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

Pharmaceutical Formulations

In certain embodiments, the present invention concerns methods and electrospun collagen-base implantable delivery systems suitable for delivery of therapeutic, diagnostic, or prophylactic agents to one or more cells or tissues of an animal, either alone, or in combination with one or more other modalities of diagnosis, prophylaxis and/or therapy. The formulation of pharmaceutically acceptable excipients and carrier solutions is well known to those of ordinary skill in the art, as is the development of suitable surgical implantation methods for using the particular membrane compositions described herein in a variety of treatment regimens, and particularly those involving bone regrowth.

Sterile injectable compositions may be prepared for storing the disclosed implantable delivery systems using appropriate solvent(s) alone, or including one or more additional ingredients using conventional methods. Generally, dispersions can be prepared by incorporating the selected sterilized active ingredient(s) into a sterile vehicle that contains the basic dispersion medium and the required other ingredients from those enumerated above. The delivery systems, membranes, and patches disclosed herein may also be formulated in solutions comprising a neutral or salt form to maintain the integrity of the systems, membranes, and patches prior to implantation.

Pharmaceutically acceptable salts include the acid addition salts (formed with the free amino groups of the protein), and which are formed with inorganic acids such as, without limitation, hydrochloric or phosphoric acids, or organic acids such as, without limitation, acetic, oxalic, tartaric, mandelic, and the like. Salts formed with the free carboxyl groups can also be derived from inorganic bases such as, without limitation, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation, and in such amount as is effective for the intended application.

The amount, implantation regimen, formulation, and preparation of the biocompatible, biodegradable drug delivery devices disclosed herein will be within the purview of the ordinary-skilled artisan having benefit of the present teaching. It is likely, however, that the administration of a particular, tissue-specific scaffold may be achieved through a one-time administration, such as, without limitation, by a single implantation of a sufficient quantity of the bioengineered drug delivery device required to provide the desired benefit. For example, an analgesic delivery patch can be implanted in a patient following a surgical procedure at or near the surgical site to reduce post-surgical pain.

It is important to note that the delivery systems and devices comprising them as disclosed herein are not in any way limited to use only in humans, or even to primates, or mammals. In certain embodiments, the disclosed implantable matrices may be employed in the amelioration of pain (e.g., “BupiPatch”) following surgical intervention; for the introduction of selected biological to selected tissue sites (e.g., “BioPatch”-mediated delivery of cells such as stem cells); or for directly administering one or more agents to the heart (e.g., “CardioPatch”) or other organs in a number of species. Such species include, but are not limited to, avian, amphibian, reptilian, and/or other animal species. Indeed the disclosed delivery systems may be formulated for veterinary use, including, without limitation, in selected livestock, exotic or domesticated animals, companion animals (including pets and such like), non-human primates, as well as zoological or otherwise captive specimens, and such like.

In accordance with certain embodiments, drug delivery devices of the present disclosure may be supplemented, further treated, or chemically modified with one or more additional bioactive molecules or biological compounds. Bioactive molecules or bioactive compounds, as used herein, refer to compounds or entities that alter, inhibit, activate, or otherwise affect one or more biological or chemical events. For example, bioactive agents may include, but are not limited to, opioid and non-opioid analgesics, antimicrobials and/or antibiotics such as erythromycin, bacitracin, neomycin, penicillin, polymycin B, tetracyclines, biomycin, chloromycetin, and streptomycins, cefazolin, ampicillin, azactam, tobramycin, clindamycin and gentamycin, etc.; immunosuppressants; anti-viral substances such as substances effective against hepatitis; enzyme inhibitors; hormones; neurotoxins; opioids; hypnotics; anti-histamines; lubricants; tranquilizers; anti-convulsants; muscle relaxants and anti-Parkinson substances; anti-spasmodics and muscle contractants including channel blockers; miotics and anti-cholinergics; anti-glaucoma compounds; anti-parasite and/or anti-protozoal compounds; modulators of cell-extracellular matrix interactions including cell growth inhibitors and antiadhesion molecules; vasodilating agents; inhibitors of DNA, RNA, or protein synthesis; anti-hypertensives; analgesics; anti-pyretics; steroidal and non-steroidal anti-inflammatory agents; anti-angiogenic factors; angiogenic factors and polymeric carriers containing such factors; anti-secretory factors; coagulants and/or clotting agents; local anesthetics; prostaglandins; amino acids; peptides; vitamins; inorganic elements, and the like.

In certain embodiments, wherein the intent is pain management, the bioactive agent is preferably an analgesic drug. In such embodiments, the implantable analgesic patch may comprise more than one analgesics, alone, or in combination with one or more additional bioactive agent(s). In such embodiments, the second agent may be a second analgesic, or alternatively, may be a growth factor, a cytokine, an extracellular matrix molecule, or a fragment or derivative thereof, one or more biocidal agents, antimicrobial agents, antibiotics, growth factors, anti-clotting agents, clotting agents, analgesics, including non-narcotic analgesics, anesthetics, including topical and/or local anesthetics, pain relievers, anti-inflammatory agents, wound repair agents, hormones, heart medications, nicotine, combinations thereof, and the like.

Exemplary cytokines, which may be employed in the preparation and implantation of one or more “BioPatches,” may include, but are not limited to, transforming growth factors (TGFs), fibroblast growth factors (FGFs), platelet derived growth factors (PDGFs), epidermal growth factors (EGFs), connective tissue activated peptides (CTAPs), cardiogenic factors, stem cell differentiating factors, osteogenic factors, as well as other biologically active analogs, fragments, and derivatives of such growth factors, polypeptides, and/or polynucleotides.

Members of the transforming growth factor (TGF) supergene family, which are multifunctional regulatory proteins, are particularly preferred. Members of the TGF supergene family include the β-transforming growth factors (for example TGF-β1, TGF-β2, TGF-β3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); Inhibins (for example, Inhibin A, Inhibin B); growth differentiating factors (for example, GDF-1); and Activins (for example, Activin A, Activin B, Activin AB). Growth factors can be isolated from native or natural sources, such as from mammalian cells, or can be prepared synthetically, such as by recombinant DNA techniques or by various chemical processes. In addition, analogs, fragments, or derivatives of these factors can be used, provided they exhibit at least some of the biological activity of the native molecule.

In exemplary analgesic/anesthetic delivery vehicles, the bioactive agent may include one or more compounds for alleviating pain, such as, and without limitation, methyl salicylate, salicylic acid, acetaminophen, morphine, fentanyl, oxycodone, hydrocodone, hydromorphone, a COX-2 inhibitor, a non-steroidal anti-inflammatory drug (NSAID), and combinations thereof. Alternatively, the bioactive agent may include one ore more anesthetics, such as benzocaine, bupivacaine, butesin picrate, chloroprocaine, ethyl chloride, fluori-methane, lidocaine HCl, mepivacaine, pramoxine HCl, combinations thereof, and the like.

Such bioactive agents are preferably contained within the drug delivery devices herein such that when implanted into the subject, the agents are released over time to provide an effective amount of the agent to the subject for the duration of treatment.

As is well known in the medical and veterinary arts, a suitable dosage for any one animal depends on many factors, including the particular animal's size, body surface area, age, the particular composition to be administered, duration of administration, location of the implant within the body, the general health of the animal, and whether other drugs or bioactive agents are being administered concurrently.

Compositions for the Preparation of Medicaments

Another important aspect of the present invention concerns methods for using the disclosed delivery systems (as well as formulations including them) in the preparation of medicaments for treating and/or ameliorating one or more symptoms of a disease, dysfunction, abnormal condition, trauma, or a genetic disorder or congenital defect in an animal, including, for example, vertebrate mammals such as humans. Use of the disclosed electrospun patches is particularly contemplated in: (a) the treatment of pain at surgical site implants; (b) delivery of one or more cells or therapeutics, such as growth factors and the like; or (c) the treatment of one or more organ defects, such as a cardiac defect, insufficiency, disorders, and the like.

Regardless of the particular application, the use of the disclosed delivery systems generally involves administration to a mammal in need thereof one or more of the disclosed patch compositions, in an amount and for a time sufficient to treat a given defect or condition. For example, for the amelioration or alleviation of pain in a tissue or wound site within or about the body of an affected mammal.

Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. The following references provide one of skill with a general definition of many of the terms used in this invention: Dictionary of Biochemistry and Molecular Biology, (2^{nd} Ed.) J. Stenesh (Ed.), Wiley-Interscience (1989); Dictionary of Microbiology and Molecular Biology (3^{rd} Ed.), P. Singleton and D. Sainsbury (Eds.), Wiley-Interscience (2007); Chambers Dictionary of Science and Technology (2^{nd} Ed.), P. Walker (Ed.), Chambers (2007); Glossary of Genetics (5^{th} Ed.), R. Rieger et al. (Eds.), Springer-Verlag (1991); and The HarperCollins Dictionary of Biology, W. G. Hale and J. P. Margham, (Eds.), HarperCollins (1991).

Although any methods and compositions similar or equivalent to those described herein can be used in the practice or testing of the present invention, the preferred methods, and compositions are described herein. For purposes of the present invention, the following terms are defined below for sake of clarity and ease of reference:

In accordance with long standing patent law convention, the words “a” and “an,” when used in this application, including the claims, denote “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, “bioactive” shall include a quality of a material such that the material has an osteointegrative potential, or in other words the ability to bond with bone. Generally, materials that are bioactive develop an adherent interface with tissues that resist substantial mechanical forces.

As used herein, a “biocompatible” material is a synthetic or natural material used to replace part of a living system or to function in intimate contact with living tissue. Biocompatible materials are intended to interface with biological systems to evaluate, treat, augment, or replace any tissue, organ, or function of the body. The biocompatible material has the ability to perform with an appropriate host response in a specific application and does not have toxic or injurious effects on biological systems. One example of a biocompatible material can be a biocompatible ceramic.

The term “biologically-functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally-equivalent to one or more of the nucleotide sequences provided herein are particularly contemplated to be useful in the practice of the methods and compositions set forth in the instant application.

As used herein, “biomimetic” shall mean a resemblance of a synthesized material to a substance that occurs naturally in a human body and which is not rejected by (e.g., does not cause an adverse reaction in) the human body.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert (s), or such like, or a combination thereof that is pharmaceutically acceptable for administration to the relevant animal or acceptable for a therapeutic or diagnostic purpose, as applicable.

As used herein, “chondrocyte” shall mean a differentiated cell responsible for secretion of extracellular matrix of cartilage. Preferably, the cells are from a compatible human donor. More preferably, the cells are from the patient (i.e., autologous cells).

As used herein, the term “DNA segment” refers to a DNA molecule that has been isolated free of total genomic DNA of a particular species. Therefore, a DNA segment obtained from a biological sample using one of the compositions disclosed herein refers to one or more DNA segments that have been isolated away from, or purified free from, total genomic DNA of the particular species from which they are obtained. Included within the term “DNA segment,” are DNA segments and smaller fragments of such segments, as well as recombinant vectors, including, for example, plasmids, cosmids, phage, viruses, and the like.

The term “effective amount,” as used herein, refers to an amount that is capable of treating or ameliorating a disease or condition or otherwise capable of producing an intended therapeutic effect.

As used herein, “fibroblast” shall mean a cell of connective tissue that secretes proteins and molecular collagen including fibrillar procollagen, fibronectin and collagenase, from which an extracellular fibrillar matrix of connective tissue may be formed. Fibroblasts synthesize and maintain the extracellular matrix of many tissues, including but not limited to connective tissue. The fibroblast cell may be mesodermally derived, and secrete proteins and molecular collagen including fibrillar procollagen, fibronectin and collagenase, from which an extracellular fibrillar matrix of connective tissue may be formed. A “fibroblast-like cell” means a cell that shares certain characteristics with a fibroblast (such as expression of certain proteins).

The terms “for example” or “e.g.,” as used herein, are used merely by way of example, without limitation intended, and should not be construed as referring only those items explicitly enumerated in the specification.

As used herein, “hard tissue” is intended to include mineralized tissues, such as bone, teeth, and cartilage. Mineralized tissues are biological tissues that incorporate minerals into soft matrices.

As used herein, a “heterologous” sequence is defined in relation to a predetermined, reference sequence, such as, a polynucleotide or a polypeptide sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, “homologous” means, when referring to polynucleotides, sequences that have the same essential nucleotide sequence, despite arising from different origins. Typically, homologous nucleic acid sequences are derived from closely related genes or organisms possessing one or more substantially similar genomic sequences. By contrast, an “analogous” polynucleotide is one that shares the same function with a polynucleotide from a different species or organism, but may have a significantly different primary nucleotide sequence that encodes one or more proteins or polypeptides that accomplish similar functions or possess similar biological activity. Analogous polynucleotides may often be derived from two or more organisms that are not closely related (e.g., either genetically or phylogenetically).

As used herein, the term “homology” refers to a degree of complementarity between two or more polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity can be determined by analyzing two or more sequences using algorithms and computer programs known in the art. Such methods may be used to assess whether a given sequence is identical or homologous to another selected sequence.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

As used herein, “implantable” or “suitable for implantation” means surgically appropriate for insertion into the body of a host, e.g., biocompatible, or having the desired design and physical properties.

As used herein, the phrase “in need of treatment” refers to a judgment made by a caregiver such as a physician or veterinarian that a patient requires (or will benefit in one or more ways) from treatment. Such judgment may made based on a variety of factors that are in the realm of a caregiver's expertise, and may include the knowledge that the patient is ill as the result of a disease state that is treatable by one or more compound or pharmaceutical compositions such as those set forth herein.

The phrases “isolated” or “biologically pure” refer to material that is substantially, or essentially, free from components that normally accompany the material as it is found in its native state.

As used herein, the term “kit” may be used to describe variations of the portable, self-contained enclosure that includes at least one set of reagents, components, or pharmaceutically-formulated compositions to conduct one or more of the assay methods of the present invention. Optionally, such kit may include one or more sets of instructions for use of the enclosed reagents, such as, for example, in a laboratory or clinical application.

“Link” or “join” refers to any method known in the art for functionally connecting one or more proteins, peptides, nucleic acids, or polynucleotides, including, without limitation, recombinant fusion, covalent bonding, disulfide bonding, ionic bonding, hydrogen bonding, electrostatic bonding, and the like.

As used herein, “matrix” shall mean a three-dimensional structure fabricated with biomaterials. The biomaterials can be biologically-derived or synthetic.

As used herein, a “medical prosthetic device,” “medical implant,” “implant,” and such like, relate to a device intended to be implanted into the body of a vertebrate animal, such as a mammal, and in particular a human. Implants in the present context may be used to replace anatomy and/or restore any function of the body. Examples of such devices include, but are not limited to, post-surgical analgesic implants, dental implants, orthopedic implants, or organ-specific applications such as the delivery of stem cells, growth factors or other suitable bioactive agents to the heart for the treatment of a cardiac defect or disorder.

In the present context, orthopedic implants, for example, includes within its scope any device intended to be implanted into the body of a vertebrate animal, in particular a mammal such as a human, for preservation and restoration of the function of the musculoskeletal system, particularly joints and bones, including the alleviation of pain in these structures.

In the present context, dental implants include any device intended to be implanted into the oral cavity of a vertebrate animal, in particular a mammal such as a human, in tooth restoration procedures. Generally, a dental implant is composed of one or several implant parts. For instance, a dental implant usually comprises a dental fixture coupled to secondary implant parts, such as an abutment and/or a dental restoration such as a crown, bridge, or denture. However, any device, such as a dental fixture, intended for implantation may alone be referred to as an implant even if other parts are to be connected thereto. Orthopedic and dental implants may also be denoted as orthopedic and dental prosthetic devices as is clear from the above. The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally-occurring animals.

As used herein, “mesh” means a network of material. The mesh may be woven synthetic fibers, non-woven synthetic fibers, nanofibers, or any combination thereof, or any material suitable for implantation into a mammal, and in particular, for implantation of electrospun collagen-based matrices within the body of a human.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally-occurring animals.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

The term “operably linked,” as used herein, refers to that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

As used herein, “osteoblast” shall mean a bone-forming cell which forms an osseous matrix in which it becomes enclosed as an osteocyte. It may be derived from mesenchymal osteoprogenitor cells. The term may also be used broadly to encompass osteoblast-like, and related, cells, such as osteocytes and osteoclasts. An “osteoblast-like cell” means a cell that shares certain characteristics with an osteoblast (such as expression of certain proteins unique to bones), but is not an osteoblast. “Osteoblast-like cells” include preosteoblasts and osteoprogenitor cells. Preferably the cells are from a compatible human donor. More preferably, the cells are from the patient (i.e., autologous cells).

As used herein, “osteointegrative” means having the ability to chemically bond to bone.

As used herein, the term “patient” (also interchangeably referred to as “host” or “subject”), refers to any host that can serve as a recipient of one or more of the therapeutic or diagnostic formulations as discussed herein. In certain aspects, the patient is a vertebrate animal, which is intended to denote any animal species (and preferably, a mammalian species such as a human being). In certain embodiments, a patient may be any animal host, including but not limited to, human and non-human primates, avians, reptiles, amphibians, bovines, canines, caprines, cavines, corvines, epines, equines, felines, hircines, lapines, leporines, lupines, murines, ovines, porcines, racines, vulpines, and the like, including, without limitation, domesticated livestock, herding or migratory animals or birds, exotics or zoological specimens, as well as companion animals, pets, or any animal under the care of a veterinary or animal medical care practitioner.

The phrase “pharmaceutically-acceptable” refers to molecular entities and compositions that preferably do not produce an allergic or similar untoward reaction when administered to a mammal, and in particular, when administered to a human. As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

The term “pharmaceutically-acceptable salt” as used herein refers to a compound of the present disclosure derived from pharmaceutically acceptable bases, inorganic or organic acids. Examples of suitable acids include, but are not limited to, hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, formic, benzoic, malonic, naphthalene-2-sulfonic, trifluoroacetic and benzenesulfonic acids. Salts derived from appropriate bases include, but are not limited to, alkali such as sodium and ammonia.

As used herein, the term “plasmid” or “vector” refers to a genetic construct that is composed of genetic material (i.e., nucleic acids). Typically, a plasmid or a vector contains an origin of replication that is functional in bacterial host cells, e.g., Escherichia coli, and selectable markers for detecting bacterial host cells including the plasmid. Plasmids and vectors of the present invention may include one or more genetic elements as described herein arranged such that an inserted coding sequence can be transcribed and translated in a suitable expression cells. In addition, the plasmid or vector may include one or more nucleic acid segments, genes, promoters, enhancers, activators, multiple cloning regions, or any combination thereof, including segments that are obtained from or derived from one or more natural and/or artificial sources.

As used herein, “polymer” means a chemical compound or mixture of compounds formed by polymerization and including repeating structural units. Polymers may be constructed in multiple forms and compositions or combinations of compositions.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures.

For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “L” isomeric form. However, residues in the “D” isomeric form may be substituted for any L-amino acid residue provided the desired properties of the polypeptide are retained.

As used herein, the terms “prevent,” “preventing,” “prevention,” “suppress,” “suppressing,” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be deemed medically useful.

As used herein, “porosity” means the ratio of the volume of interstices of a material to a volume of a mass of the material.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about two to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. Polypeptides and proteins of the present invention also include polypeptides and proteins that are or have been post-translationally modified, and include any sugar or other derivative(s) or conjugate(s) added to the backbone amino acid chain.

“Purified,” as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

The term “recombinant” indicates that the material (e.g., a polynucleotide or a polypeptide) has been artificially or synthetically (non-naturally) altered by human intervention. The alteration can be performed on the material within or removed from, its natural environment, or native state. Specifically, e.g., a promoter sequence is “recombinant” when it is produced by the expression of a nucleic acid segment engineered by the hand of man. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant AAV virus, is produced by the expression of a recombinant nucleic acid.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “RNA segment” refers to an RNA molecule that has been isolated free of total cellular RNA of a particular species. Therefore, RNA segments can refer to one or more RNA segments (either of native or synthetic origin) that have been isolated away from, or purified free from, other RNAs. Included within the term “RNA segment,” are RNA segments and smaller fragments of such segments.

The term “a sequence essentially as set forth in SEQ ID NO:X” means that the sequence substantially corresponds to a portion of SEQ ID NO:X and has relatively few nucleotides (or amino acids in the case of polypeptide sequences) that are not identical to, or a biologically functional equivalent of, the nucleotides (or amino acids) of SEQ ID NO:X. The term “biologically functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally equivalent to one or more of the nucleotide sequences provided herein are particularly contemplated to be useful in the practice of the invention.

Suitable standard hybridization conditions for nucleic acids for use in the present invention include, for example, hybridization in 50% formamide, 5× Denhardt's solution, 5×SSC, 25 mM sodium phosphate, 0.1% SDS and 100 μg/mL of denatured salmon sperm DNA at 42° C. for 16 hr followed by 1 hr sequential washes with 0.1x SSC, 0.1% SDS solution at 60° C. to remove the desired amount of background signal. Lower stringency hybridization conditions for the present invention include, for example, hybridization in 35% formamide, 5× Denhardt's solution, 5×SSC, 25 mM sodium phosphate, 0.1% SDS and 100 μg/mL denatured salmon sperm DNA or E. coli DNA at 42° C. for 16 hr followed by sequential washes with 0.8×SSC, 0.1% SDS at 55° C. Those of ordinary skill in the art will recognize that such hybridization conditions can be readily adjusted to obtain the desired level of stringency for a particular application.

As used herein, “scaffold,” relates to an open porous structure. A scaffold may comprise one or more building materials to create the structure of the scaffold. Additionally, the scaffold may further comprise other substances, such as one or more biologically active molecules or such like.

As used herein, “soft tissue” is intended to include tissues that connect, support, or surround other structures and organs of the body, not being bone. Soft tissue includes ligaments, tendons, fascia, skin, fibrous tissues, fat, synovial membranes, epithelium, muscles, nerves and blood vessels.

As used herein, “stem cell” means an unspecialized cell that has the potential to develop into many different cell types in the body, such as mesenchymal osteoprogenitor cells, osteoblasts, osteocytes, osteoclasts, chondrocytes, and chondrocyte progenitor cells. Preferably, the cells are from a compatible human donor. More preferably, the cells are from the patient (i.e., autologous cells).

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, about 2, about 3, about 4, about 5, about 6, about 7, about 8, about 9, or about 10 or so base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary nucleic acid sequences will be greater than about 80 percent complementary (or “% exact-match”) to a corresponding nucleic acid target sequence to which the nucleic acid specifically binds, and will, more preferably be greater than about 85 percent complementary to the corresponding target sequence to which the nucleic acid specifically binds. In certain aspects, as described above, it will be desirable to have even more substantially complementary nucleic acid sequences for use in the practice of the invention, and in such instances, the nucleic acid sequences will be greater than about 90 percent complementary to the corresponding target sequence to which the nucleic acid specifically binds, and may in certain embodiments be greater than about 95 percent complementary to the corresponding target sequence to which the nucleic acid specifically binds, and even up to and including about 96%, about 97%, about 98%, about 99%, and even about 100% exact match complementary to all or a portion of the target sequence to which the designed nucleic acid specifically binds.

Percent similarity or percent complementary of any of the disclosed nucleic acid sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

The terms “substantially corresponds to,” “substantially homologous,” or “substantial identity,” as used herein, denote characteristics of a nucleic acid or an amino acid sequence, wherein a selected nucleic acid sequence or a selected amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

As used herein, “synthetic” shall mean that the material is not of a human or animal origin.

The term “therapeutically-practical period” means the period of time that is necessary for one or more active agents to be therapeutically effective. The term “therapeutically-effective” refers to reduction in severity and/or frequency of one or more symptoms, elimination of one or more symptoms and/or underlying cause, prevention of the occurrence of symptoms and/or their underlying cause, and the improvement or a remediation of damage.

A “therapeutic agent” may be any physiologically or pharmacologically active substance that may produce a desired biological effect in a targeted site in a subject. The therapeutic agent may be an anaesthetic, an analgesic, a chemotherapeutic agent, an immunosuppressive agent, a cytokine, a cytotoxic agent, a therapeutic polypeptide or polynucleotide, a proteolytic or nucleolytic compound, a radioactive isotope, a receptor, an enzyme, or a pro-drug activating enzyme, which may be naturally occurring, produced by synthetic or recombinant methods, or a combination thereof. Drugs that are affected by classical multidrug resistance, such as vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) may have particular utility as the therapeutic agent. Cytokines may be also used as the therapeutic agent. Examples of such cytokines are lymphokines, monokines, and traditional polypeptide hormones. A cancer chemotherapy agent may also be delivered using one or more of the disclosed scaffolds or matrices. For a more detailed description of anticancer agents and other therapeutic agents, those skilled in the art are referred to any number of instructive manuals including, but not limited to, the Physician's Desk Reference and the work by Hardman and Limbird (2001).

As used herein, a “transcription factor recognition site” and a “transcription factor binding site” refer to a polynucleotide sequence(s) or sequence motif(s), which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted based on known consensus sequence motifs, or by other methods known to those of ordinary skill in the art.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis-sequences that are necessary for efficient transcription and translation (e.g., polyadenylation site(s), mRNA stability controlling sequence(s), etc.

As used herein, the term “transformation” is intended to generally describe a process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

As used herein, the term “transformed cell” is intended to mean a host cell whose nucleic acid complement has been altered by the introduction of one or more exogenous polynucleotides into that cell.

“Treating” or “treatment of” as used herein, refers to providing any type of medical or surgical management to a subject. Treating can include, but is not limited to, administering a composition comprising a therapeutic agent to a subject. “Treating” includes any administration or application of a compound or composition of the invention to a subject for purposes such as curing, reversing, alleviating, reducing the severity of, inhibiting the progression of, or reducing the likelihood of a disease, disorder, or condition or one or more symptoms or manifestations of a disease, disorder, or condition. In certain aspects, the compositions of the present invention may also be administered prophylactically, i.e., before development of any symptom or manifestation of the condition, where such prophylaxis is warranted. Typically, in such cases, the subject will be one that has been diagnosed for being “at risk” of developing such a disease or disorder, either as a result of familial history, medical record, or the completion of one or more diagnostic or prognostic tests indicative of a propensity for subsequently developing such a disease or disorder.

The tern “vector,” as used herein, refers to a nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

In certain embodiments, it will be advantageous to deliver one or more nucleic acid segments using a delivery system disclosed herein, including, for example, in combination with an appropriate detectable marker (i.e., a “label,”). A wide variety of appropriate indicator compounds and compositions are known in the art for labeling polynucleotides and polypeptides, including, without limitation, fluorescent, radioactive, enzymatic or other ligands, such as avidin/biotin, etc., which are capable of being detected in a suitable assay or observed in situ. In certain embodiments, it may be desirable to include one or more fluorescent labels or an enzyme tag such as urease, alkaline phosphatase or peroxidase, instead of radioactive or other environmentally less-desirable reagents as part of the delivery system.

In the case of enzyme tags, colorimetric, chromogenic, or fluorogenic indicator substrates are known that can be employed to provide a method for detecting the sample that is visible to the human eye, or by analytical methods such as scintigraphy, fluorimetry, spectrophotometry, and the like, to identify specific hybridization with samples containing one or more complementary or substantially complementary nucleic acid sequences. In the case of so-called “multiplexing” assays, where two or more labeled probes are detected either simultaneously or sequentially, it may be desirable to label a first oligonucleotide probe with a first label having a first detection property or parameter (for example, an emission and/or excitation spectral maximum), which also labeled a second oligonucleotide probe with a second label having a second detection property or parameter that is different (i.e., discreet or discernible from the first label. The use of multiplexing assays, particularly in the context of genetic amplification/detection protocols are well-known to those of ordinary skill in the molecular genetic arts.

Biological Functional Equivalents

Modification and changes may be made in the structure of a nucleic acid, or to vectors comprising it, as well as to mRNAs, polypeptides, or therapeutic agents encoded by them and still obtain functional systems that contain one or more therapeutic agents with desirable characteristics. As mentioned above, it is often desirable to introduce one or more mutations into a specific polynucleotide sequence. In certain circumstances, the resulting encoded polypeptide sequence is altered by this mutation, or in other cases, the sequence of the polypeptide is unchanged by one or more mutations in the encoding polynucleotide.

When it is desirable to alter the amino acid sequence of a polypeptide to create an equivalent, or even an improved, second-generation molecule, the amino acid changes may be achieved by changing one or more of the codons of the encoding DNA sequence, according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions or corresponding DNA sequences which encode said peptides without appreciable loss of their biological utility or activity.

TABLE 1

| | AMINO | | | | | | |
|---|---|---|---|---|---|---|---|
| | ACIDS | CODONS | | | | | |
| Alanine | Ala | GCA | GCC | GCG | GCU | | |
| Cysteine | Cys | UGC | UGU | | | | |
| Aspartic acid | Asp | GAC | GAU | | | | |
| Glutamic acid | Glu | GAA | GAG | | | | |
| Phenylalanine | Phe | UUC | UUU | | | | |
| Glycine | Gly | GGA | GGC | GGG | GGU | | |
| Histidine | His | CAC | CAU | | | | |
| Isoleucine | Ile | AUA | AUC | AUU | | | |
| Lysine | Lys | AAA | AAG | | | | |
| Leucine | Leu | UUA | UUG | CUA | CUC | CUG | CUU |
| Methionine | Met | AUG | | | | | |
| Asparagine | Asn | AAC | AAU | | | | |
| Proline | Pro | CCA | CCC | CCG | CCU | | |
| Glutamine | Gln | CAA | CAG | | | | |
| Arginine | Arg | AGA | AGG | CGA | CGC | CGG | CGU |
| Serine | Ser | AGC | AGU | UCA | UCC | UCG | UCU |
| Threonine | Thr | ACA | ACC | ACG | ACU | | |
| Valine | Val | GUA | GUC | GUG | GUU | | |
| Tryptophan | Trp | UGG | | | | | |
| Tyrosine | Tyr | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, specifically incorporated herein in its entirety by express reference thereto). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index based on its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively based on hydrophilicity. U.S. Pat. No. 4,554,101 (specifically incorporated herein in its entirety by express reference thereto), states that the greatest local average hydrophilicity of a protein, as governed by the hydrophilicity of its adjacent amino acids, correlates with a biological property of the protein.

As detailed in U.S. Pat. No. 4,554,101 (specifically incorporated herein in its entirety by express reference thereto), the following hydrophilicity values have been assigned to amino acid residues: arginine (+3.0); lysine (+3.0); aspartate (+3.0 ±1); glutamate (+3.0 ±1); serine (+0.3); asparagine (+0.2); glutamine (+0.2); glycine (0); threonine (−0.4); proline (−0.5 ±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take one or more of the foregoing characteristics into consideration are well known to those of ordinary skill in the art, and include arginine and lysine; glutamate and aspartate; serine and threonine; glutamine and asparagine; and valine, leucine and isoleucine.

The section headings used throughout are for organizational purposes only and are not to be construed as limiting the subject matter described. All documents, or portions of documents, cited in this application (including, but not limited to, patents, patent applications, articles, books, and treatises) are expressly incorporated herein in their entirety by express reference thereto. In the event that one or more of the incorporated literature and similar materials defines a term in a manner that contradicts the definition of that term in this application, this application controls.

### EXAMPLES

The Examples attached hereto are included to demonstrate preferred embodiments of the invention. It should be appreciated by those of skill in the art that the techniques disclosed in the accompany examples represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Bupipatch—A New Implantable Tool to Treat Incisional Pain

NANOMEDICINE: A Promising Approach to Pain Therapy

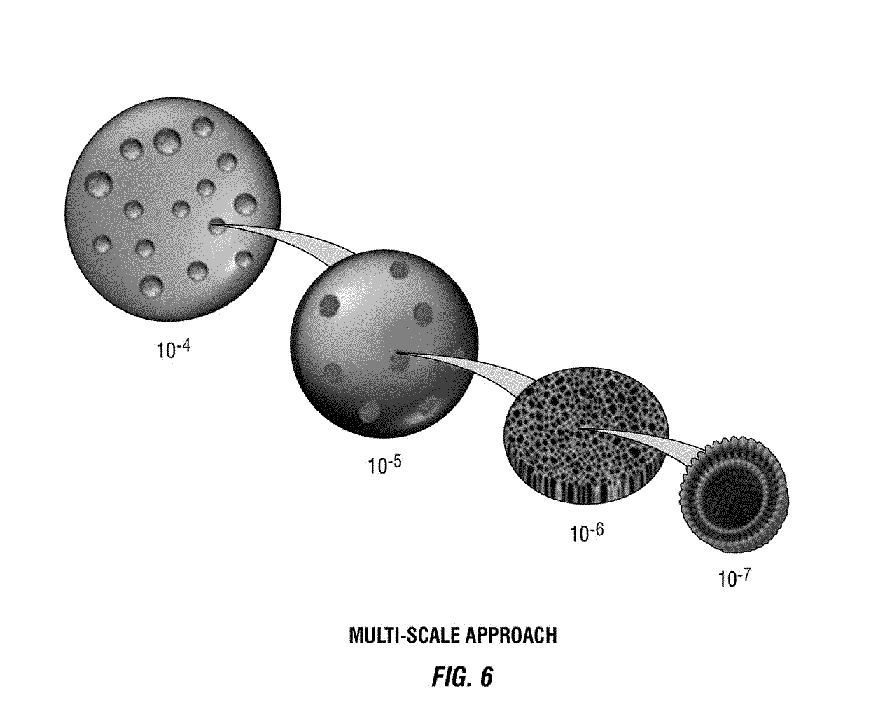

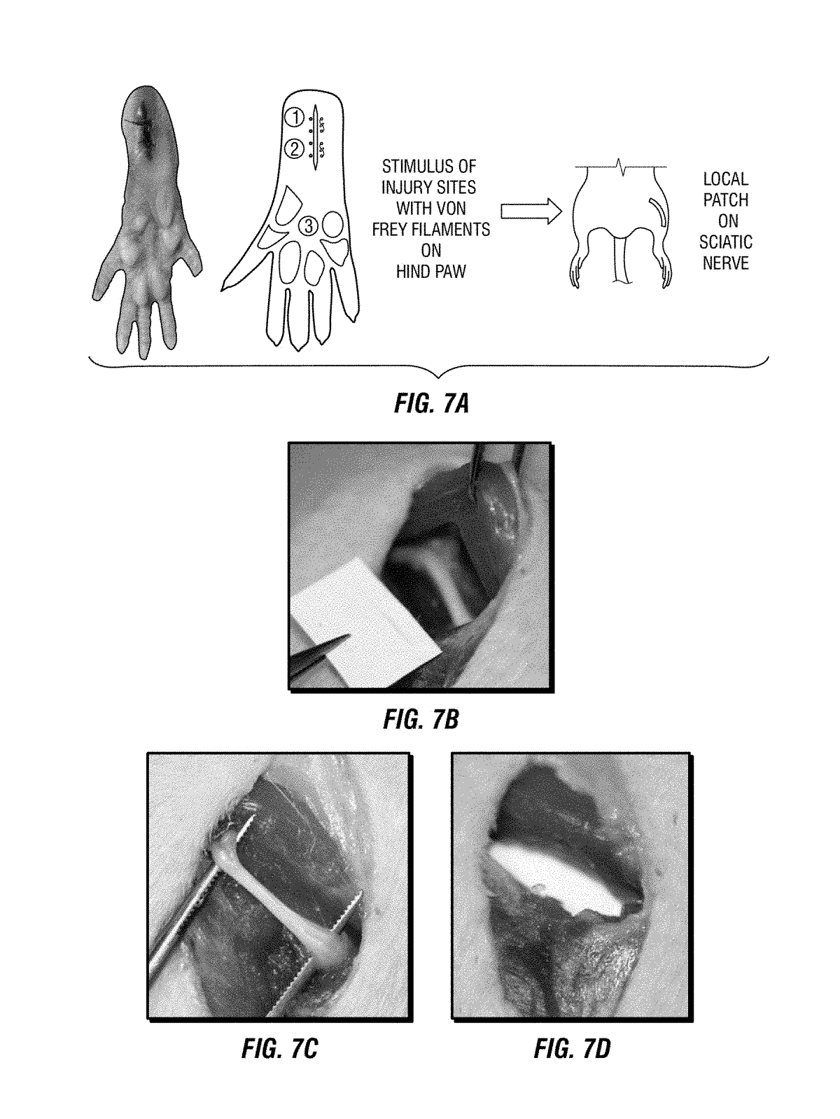

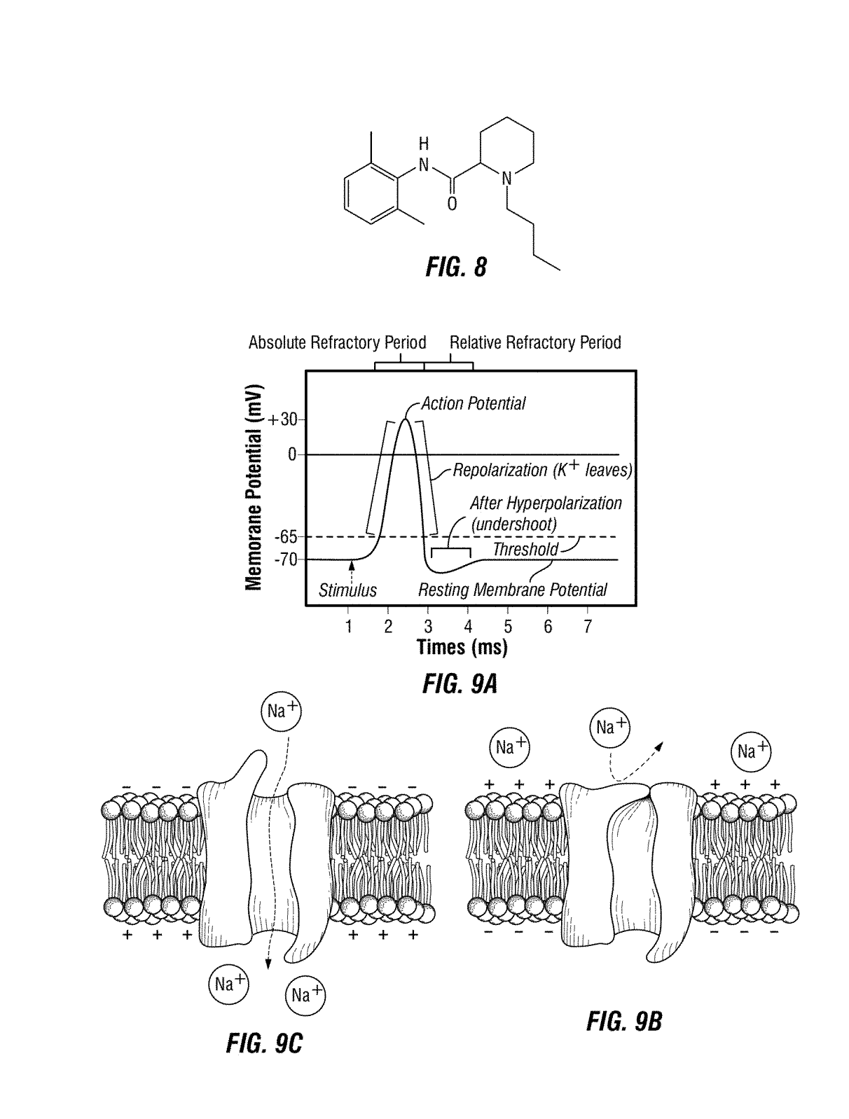

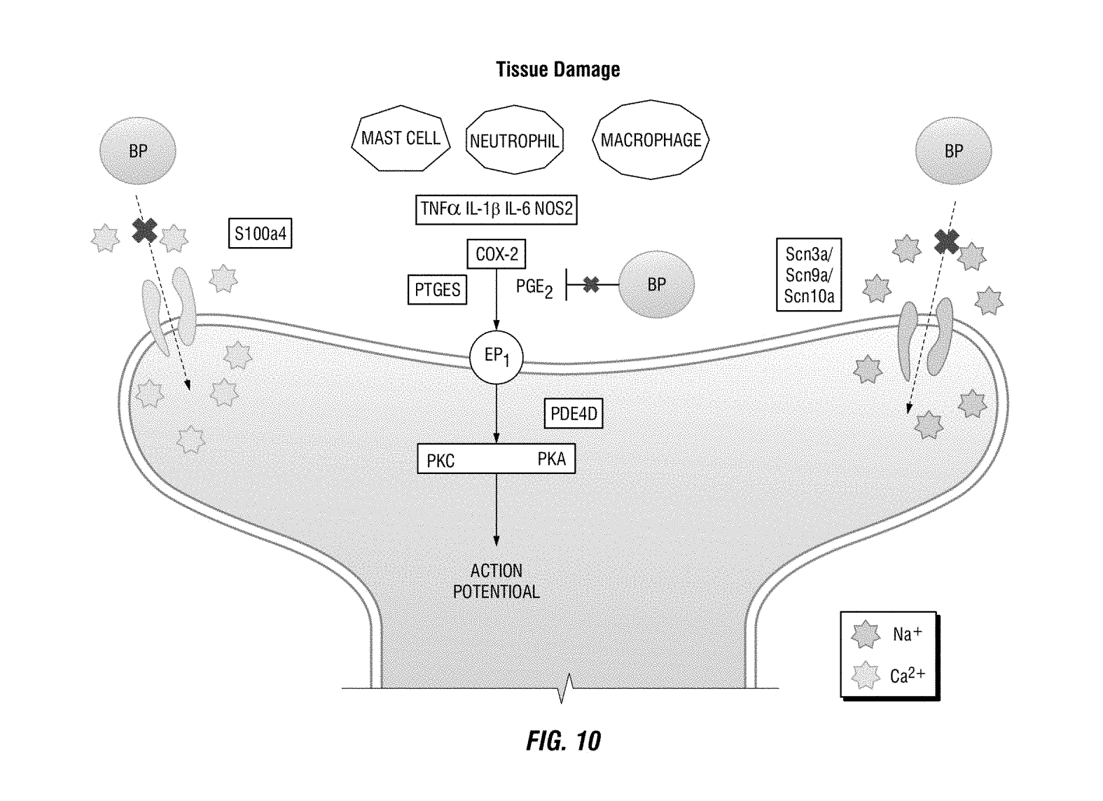

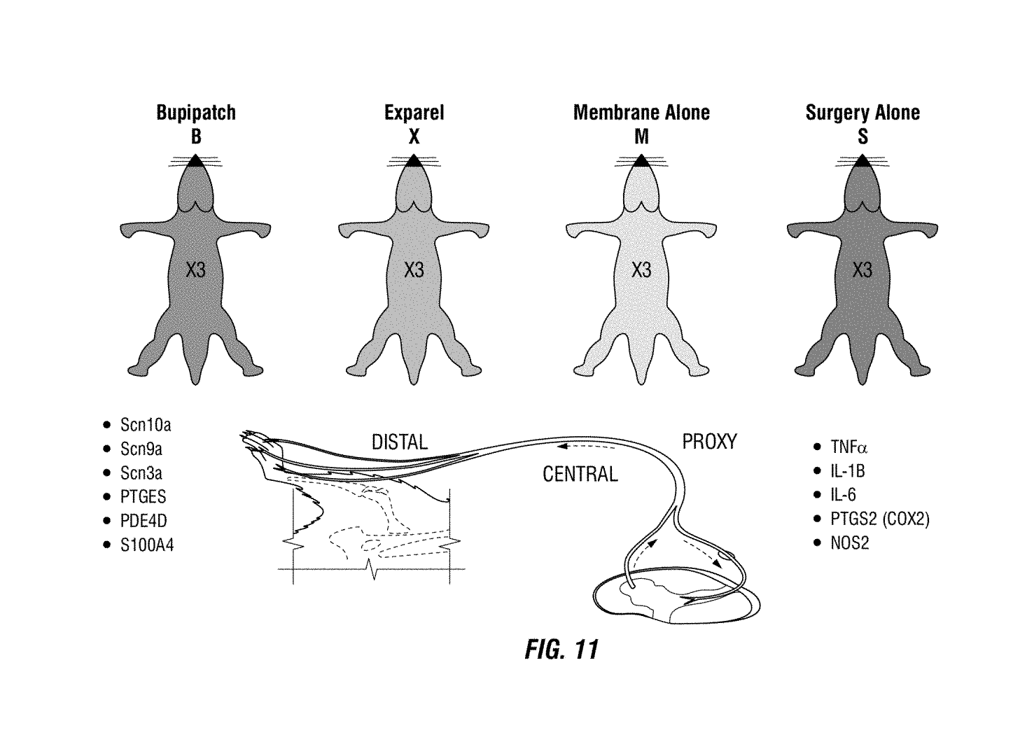

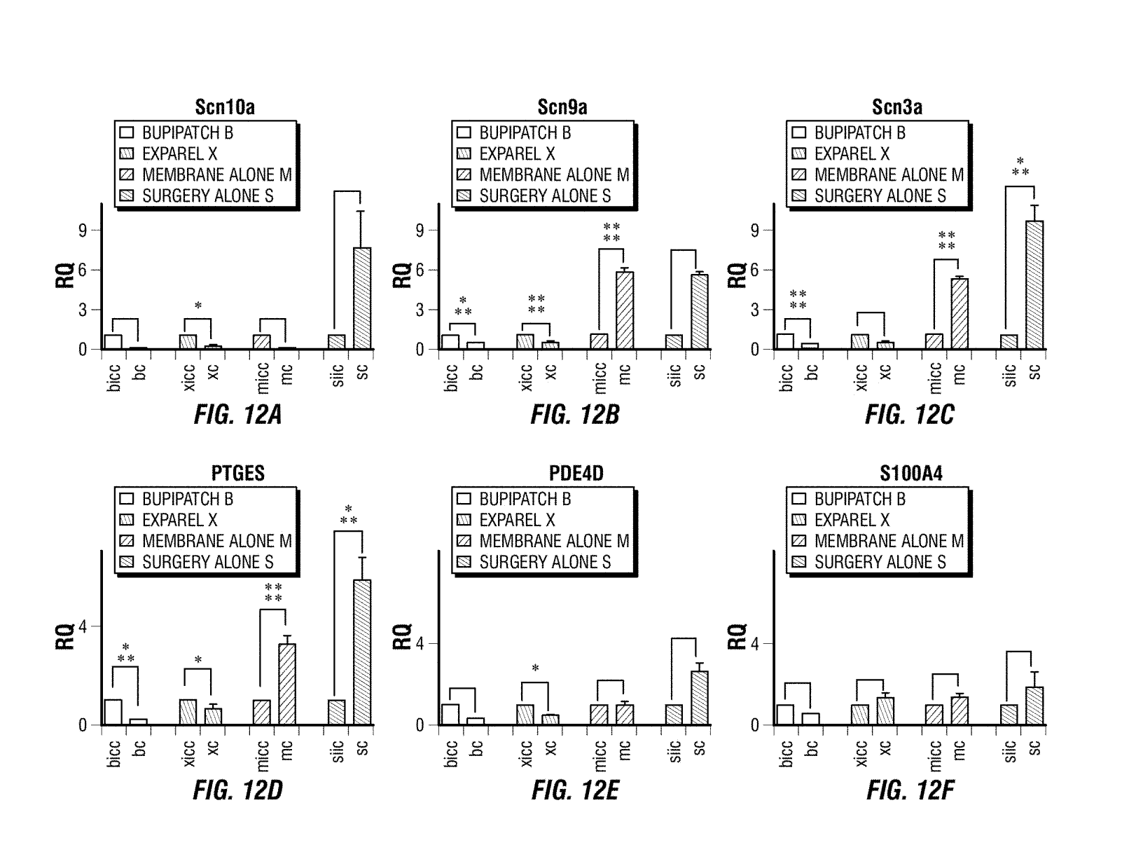

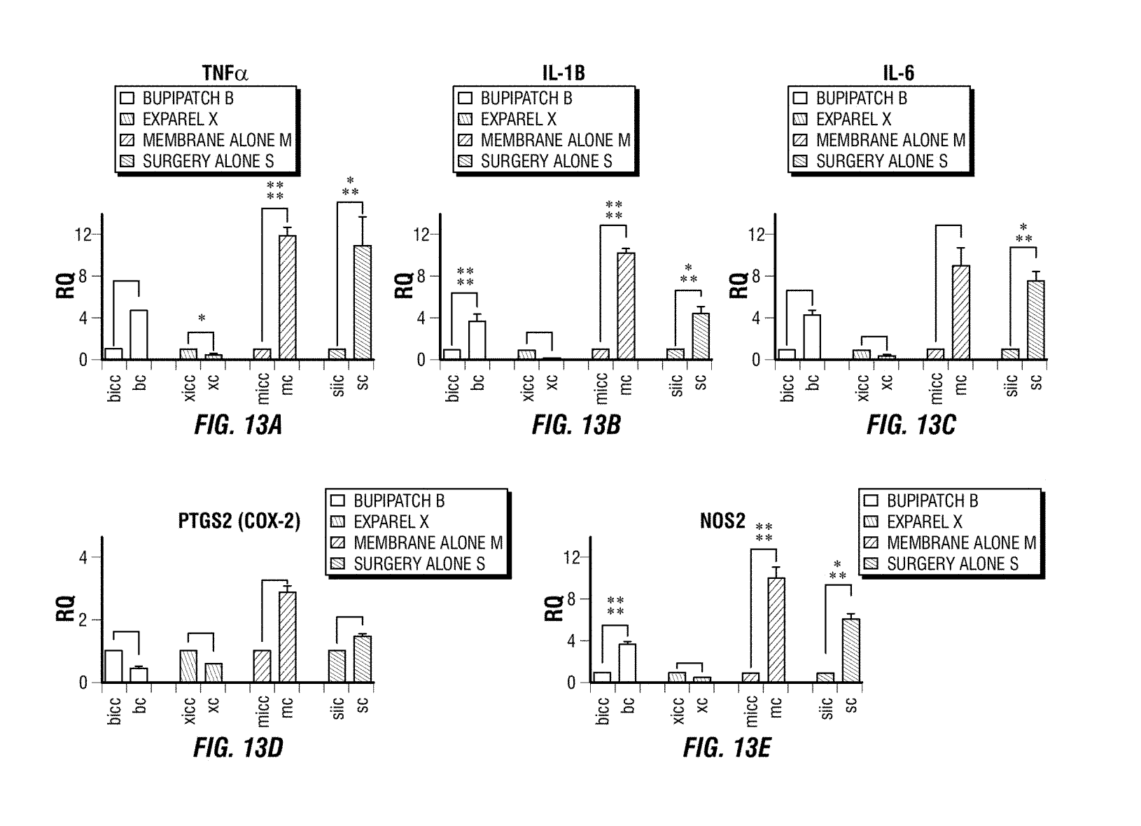

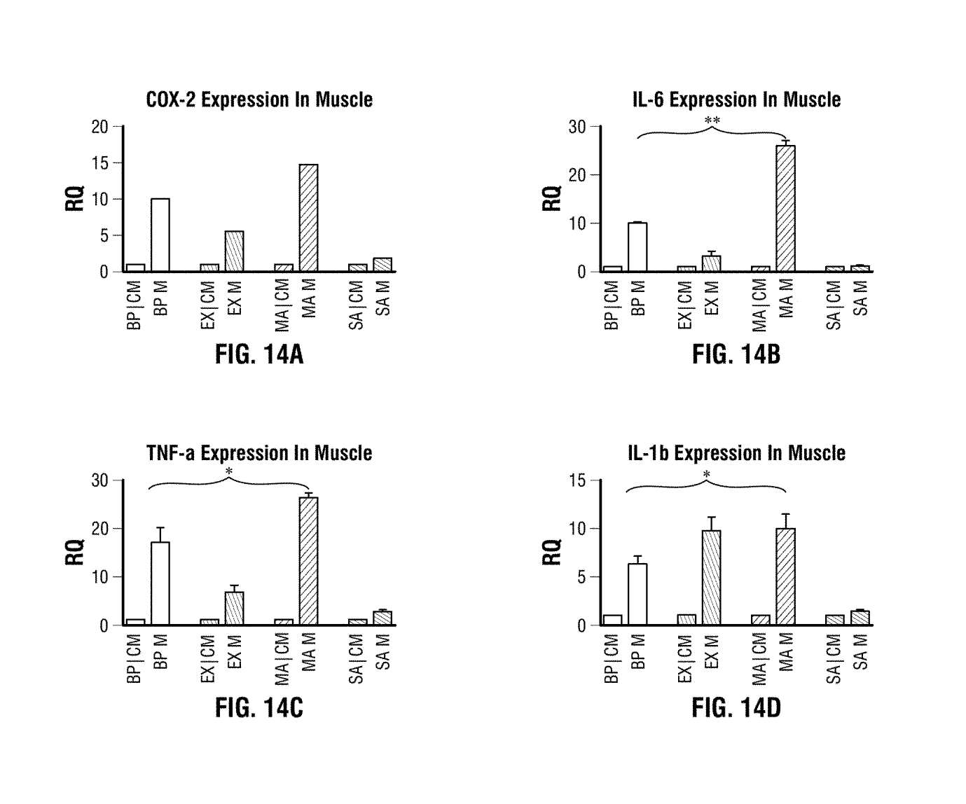

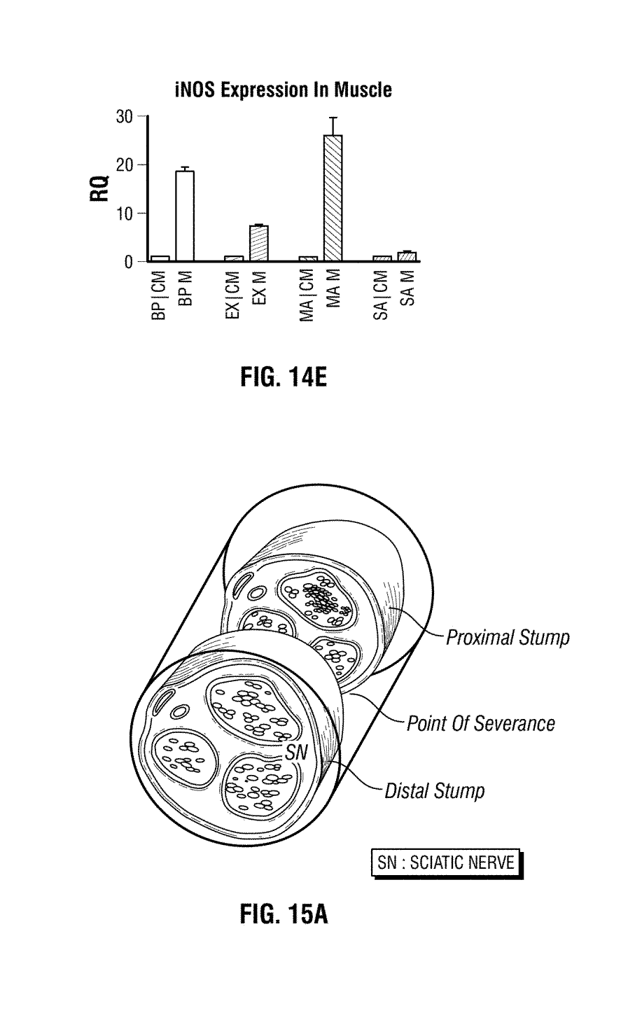

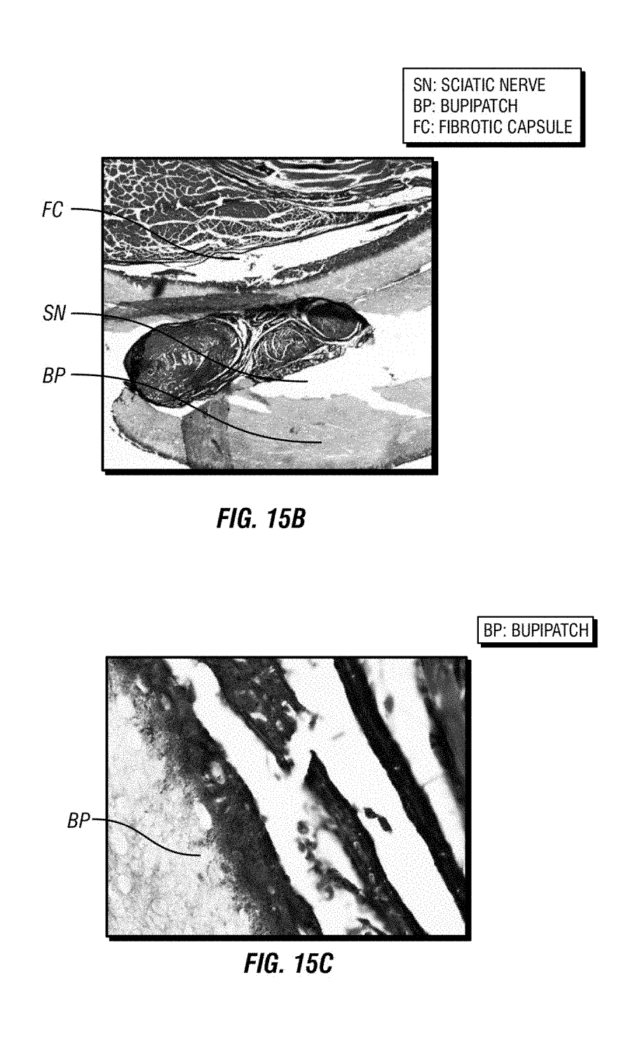

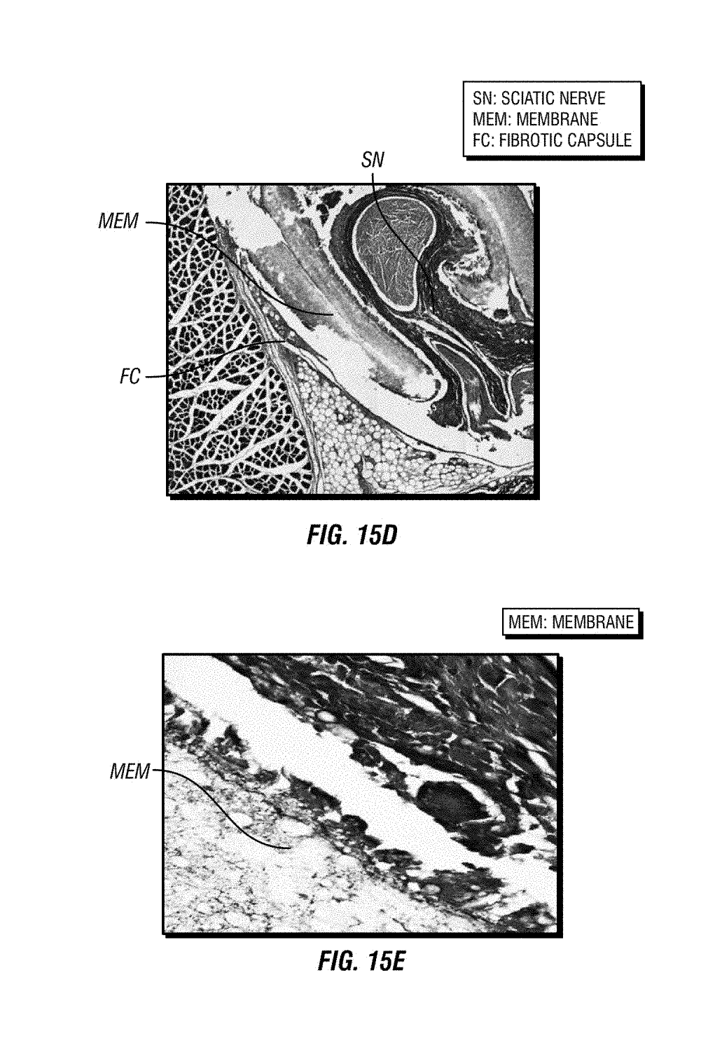

The numbers of non-opioid therapies currently available to treat pain are limited in both number and in duration of efficacy, making this field attractive as an option to apply our nanotechnologies for drug delivery. FIG. 6 shows a multi-scale approach.

Pain severely limits the full return to daily activities even in those with successful surgical outcome. 30-45% report moderate to severe pain for up to 7 days; 80% does NOT return to work by postop day 7. The number of non-opioid therapies currently available to treat pain is limited both in number and in duration of efficacy, making this field attractive as an option to apply our nanotechnologies for drug delivery.

The advantages of nanotechnologies for drug delivery include:

Biomimetic/Bioactive→Reduced inflammation;

Localized/Targeted→Reduced side effects;

Tunable→Longer controlled releases kinetics; and

Biocompatible and Biodegradable→Safe application with no toxicity.

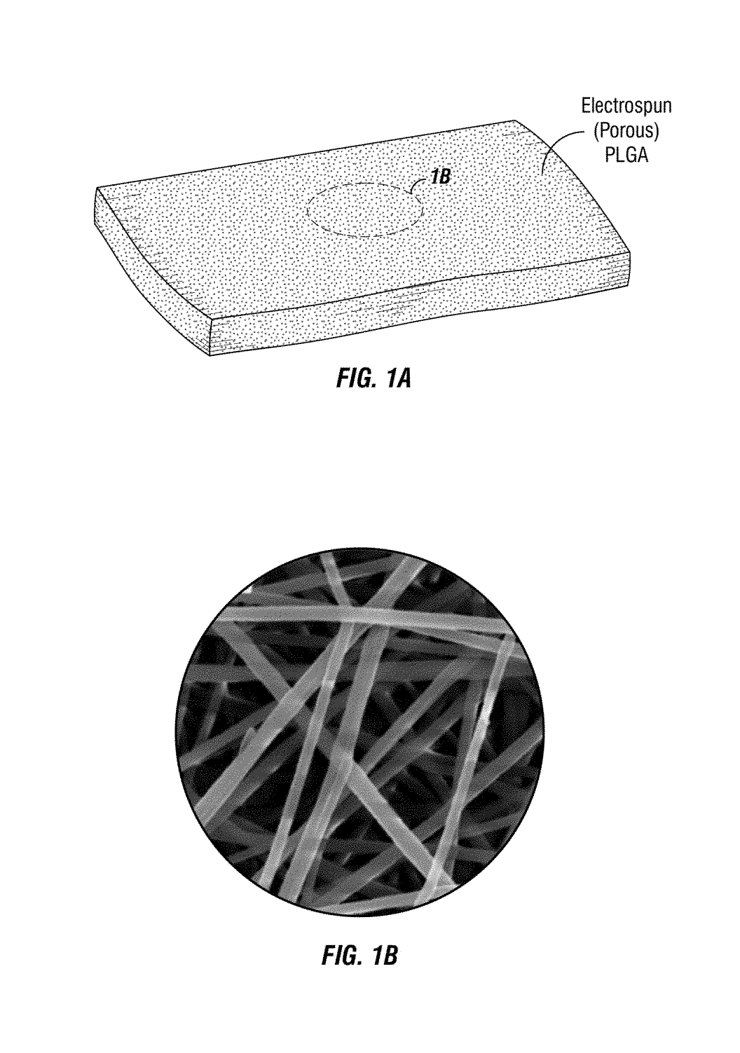

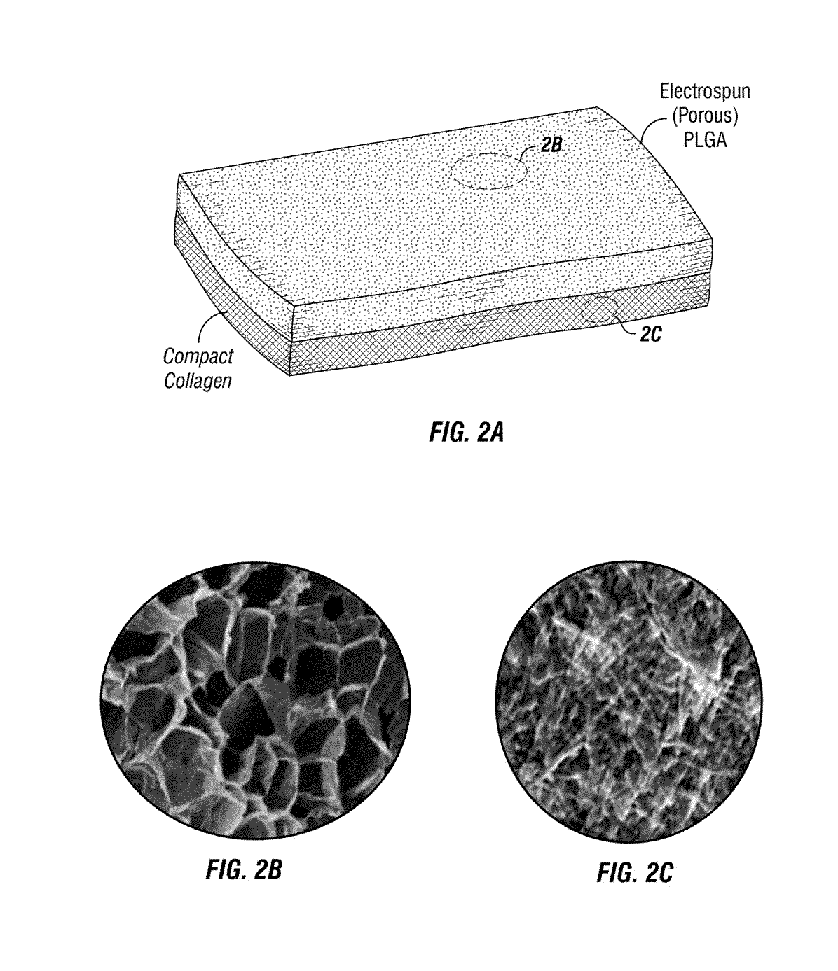

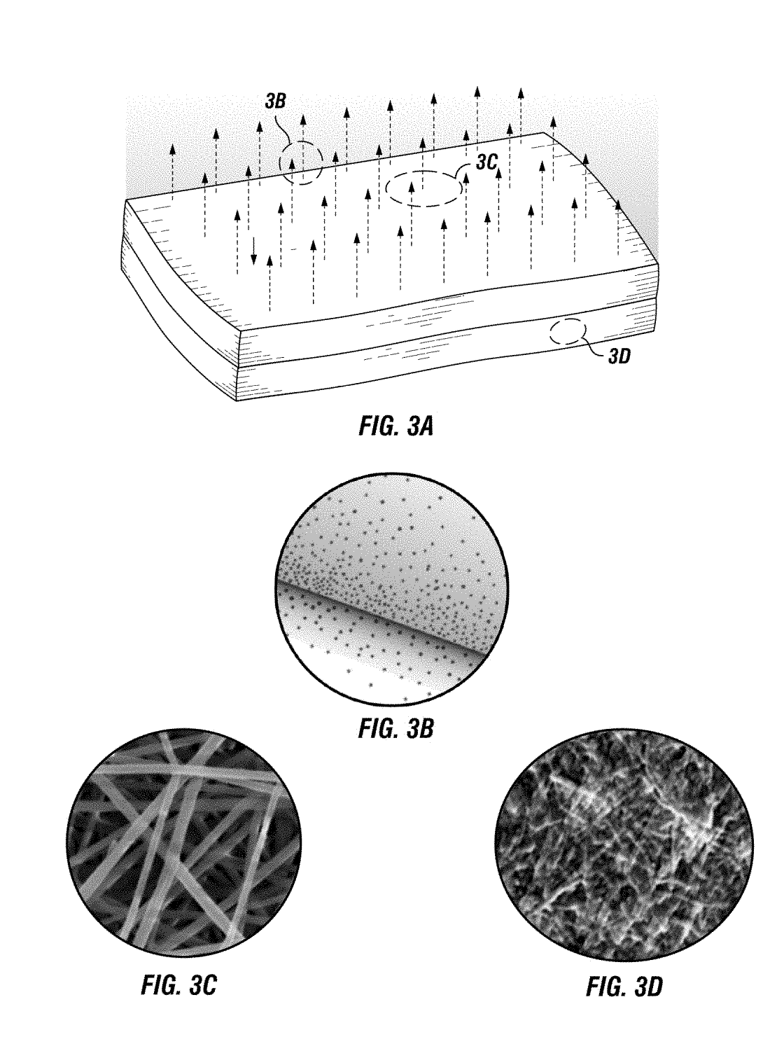

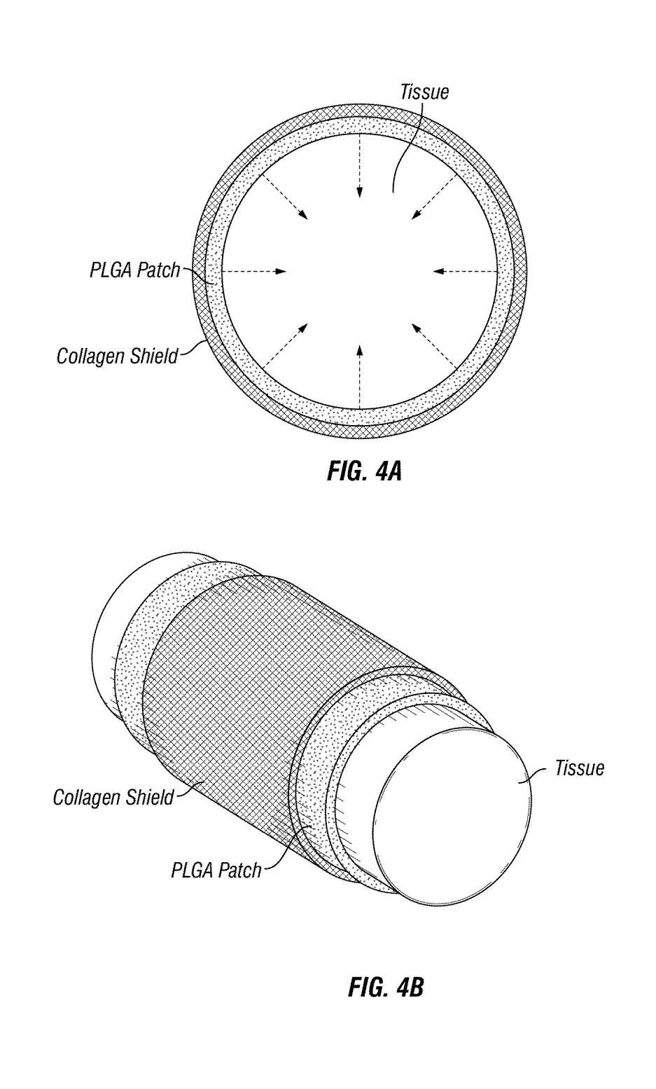

The active release of bupivacaine from a “bupipatch” analgesic patch (FIG. 1A, FIG. 1B, FIG. 2A, and FIG. 2B) will modulate pain related genes (up to seven days) after surgery.

Amino-amide local anesthetics block ion-dependent channels, block sodium ion influx into nerve cells, with no depolarization.

Bupivacaine lasts longer than other local anesthetics (lidocaine)

Bupipatch induced the downregulation of sodium channel genes in sciatic nerve in comparison to Exparel®.

The effect of Bupipatch was due to the prolonged release of bupivacaine.

The release of bupivacaine decreased the inflammation induced by the implant.

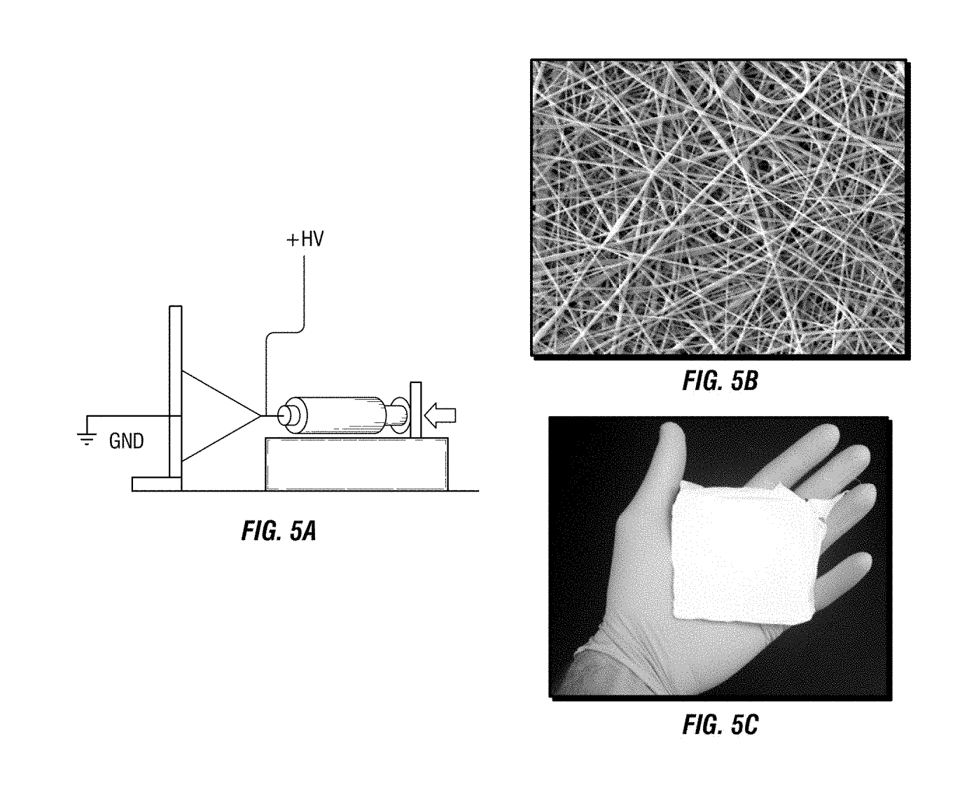

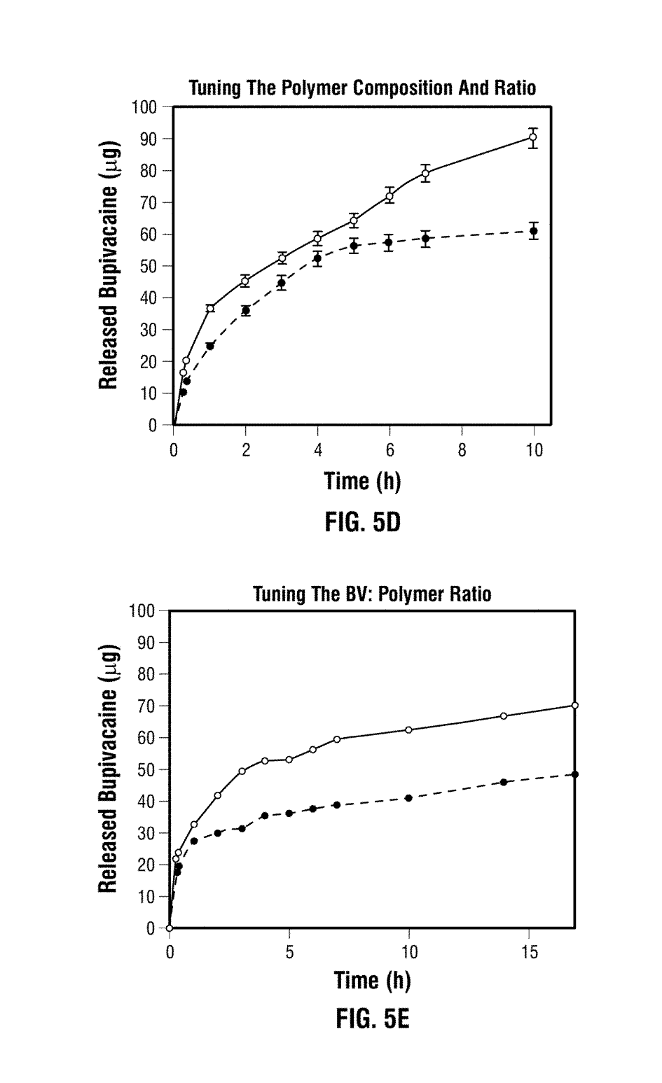

FIG. 5A, FIG. 5B, FIG. 5C, FIG. 5D, and FIG. 5E show the clinical Relevance: Postoperative Acute Pain is an issue: Pain severely limits the full return to daily activities even in those with successful surgical outcome. 30-45% report moderate to severe pain for up to 7 days. 80% does NOT return to work by postop day 7.

### Example 2

### Biopatch—Electrospun Patches for the Controlled Release of Growth Factors to Enhance Vascularization

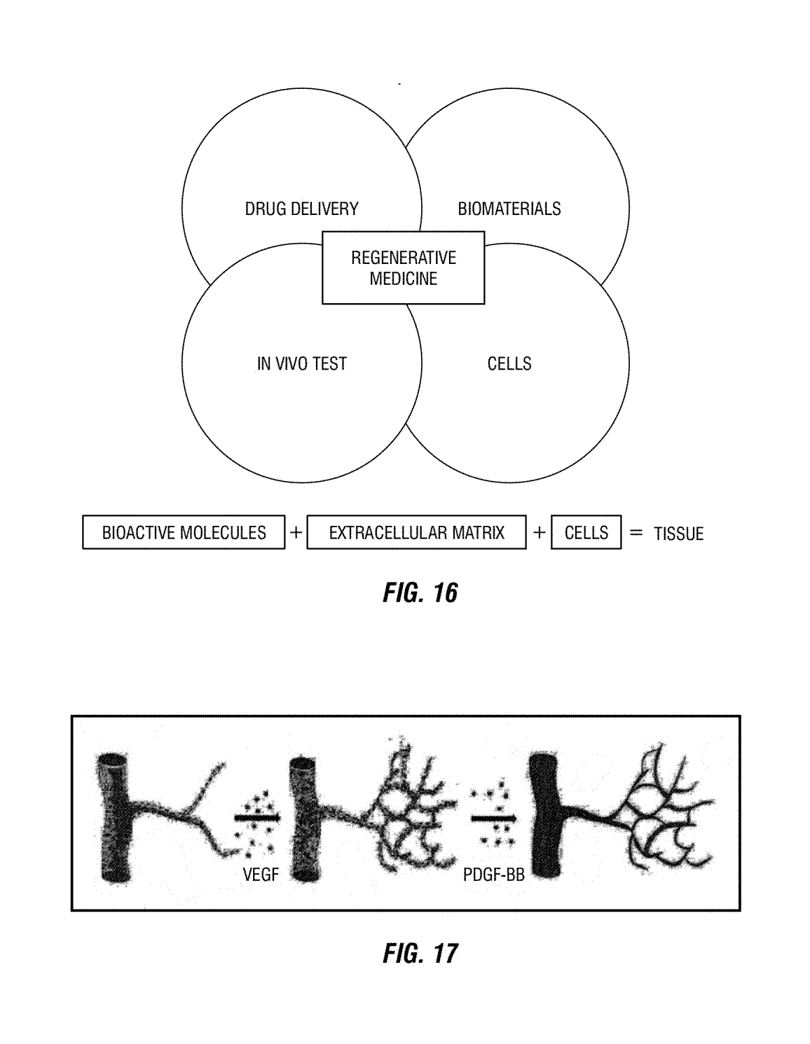

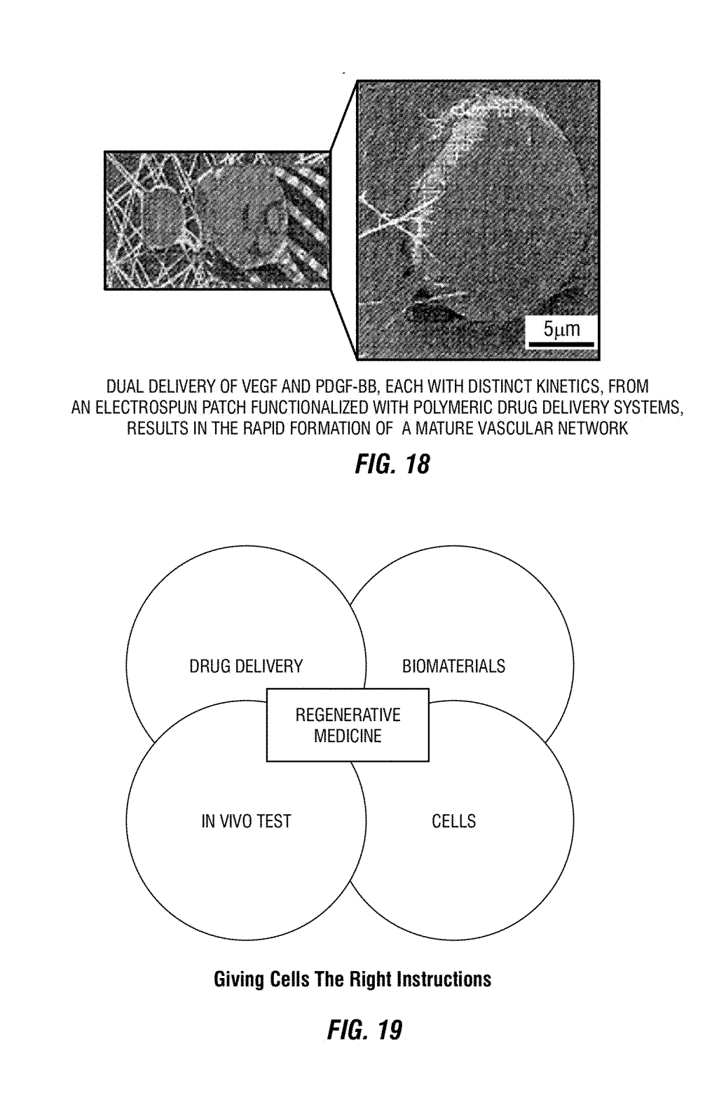

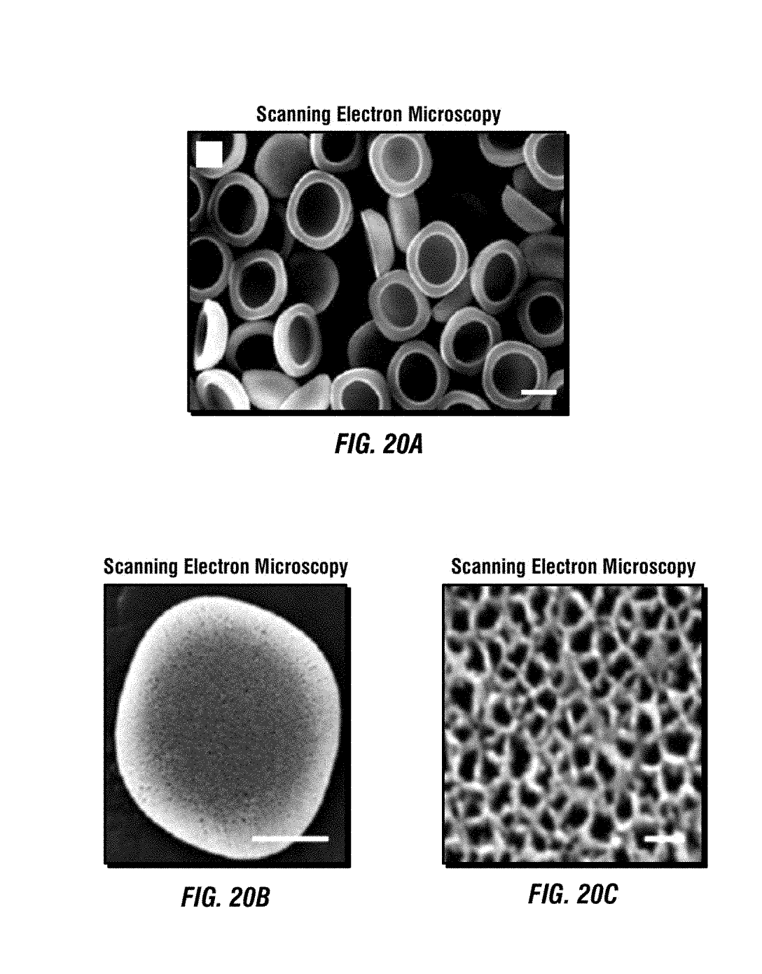

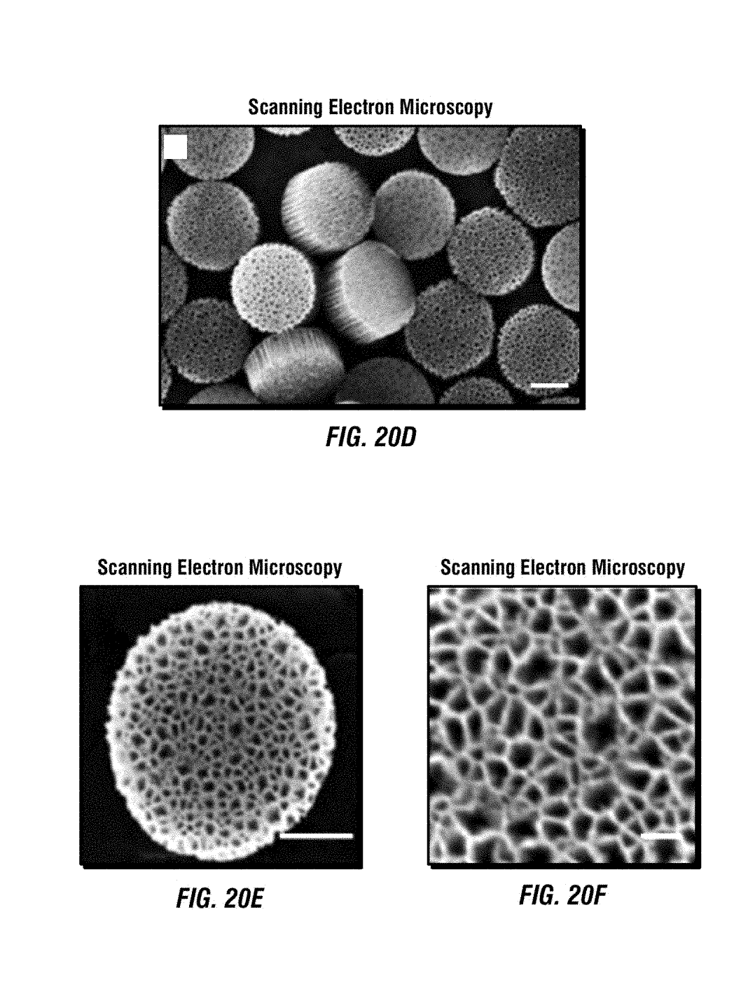

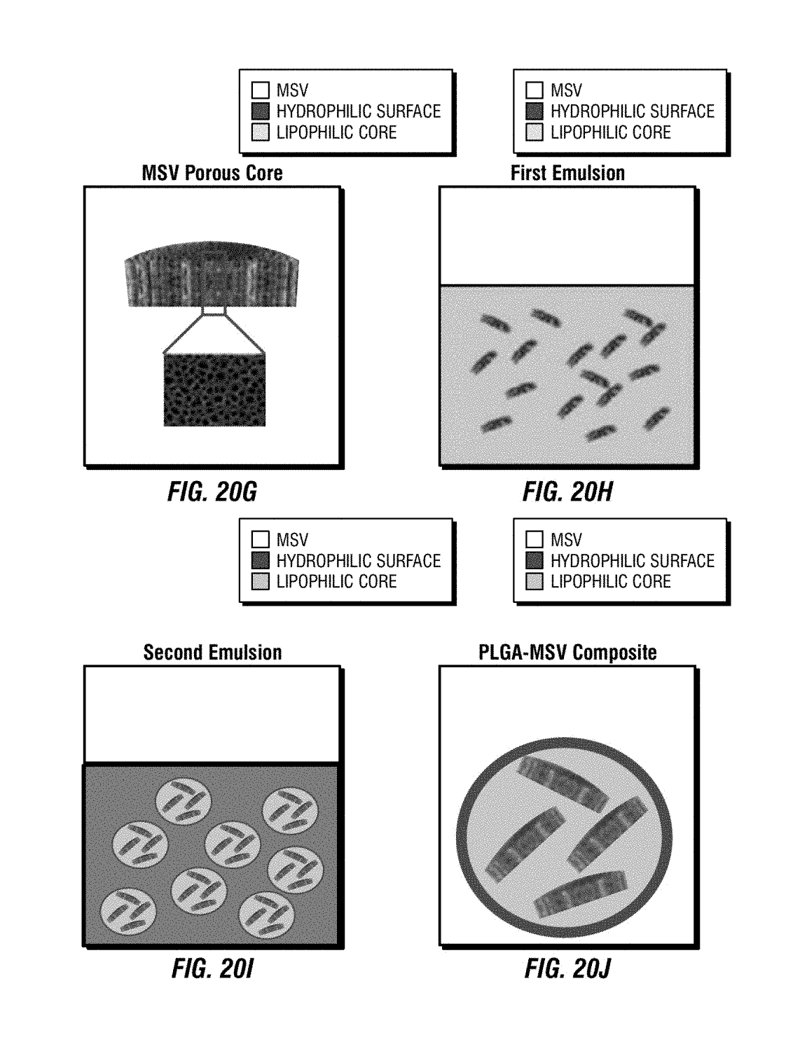

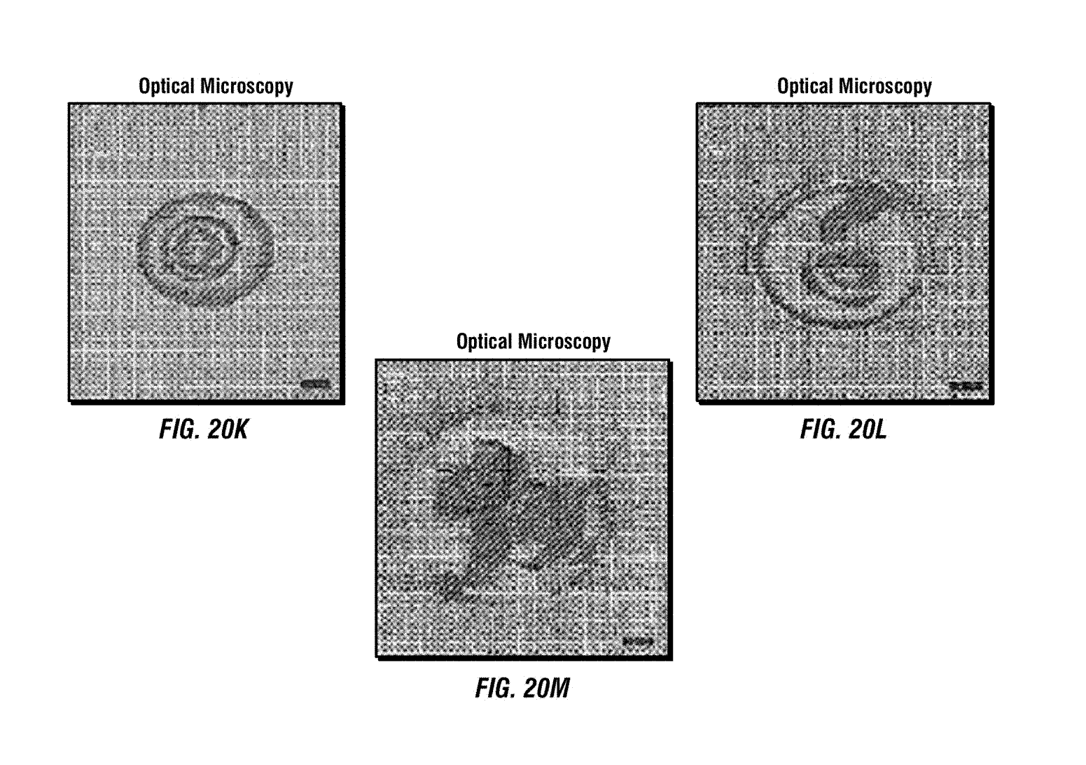

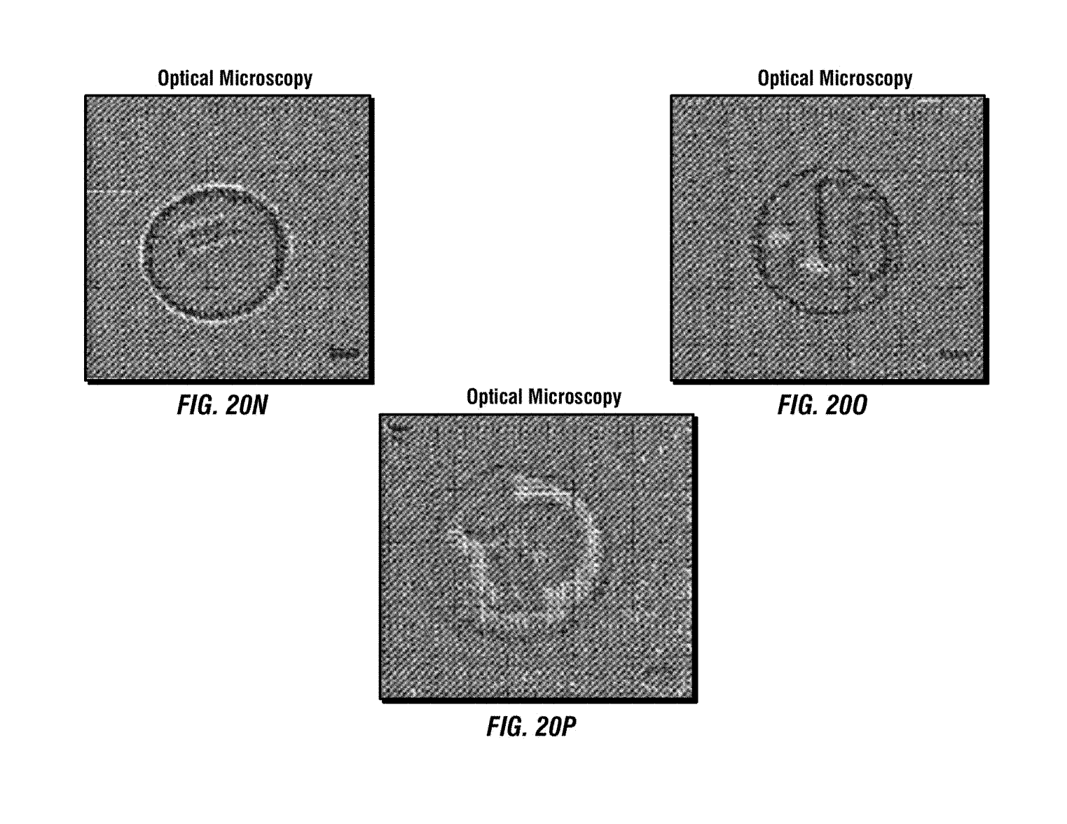

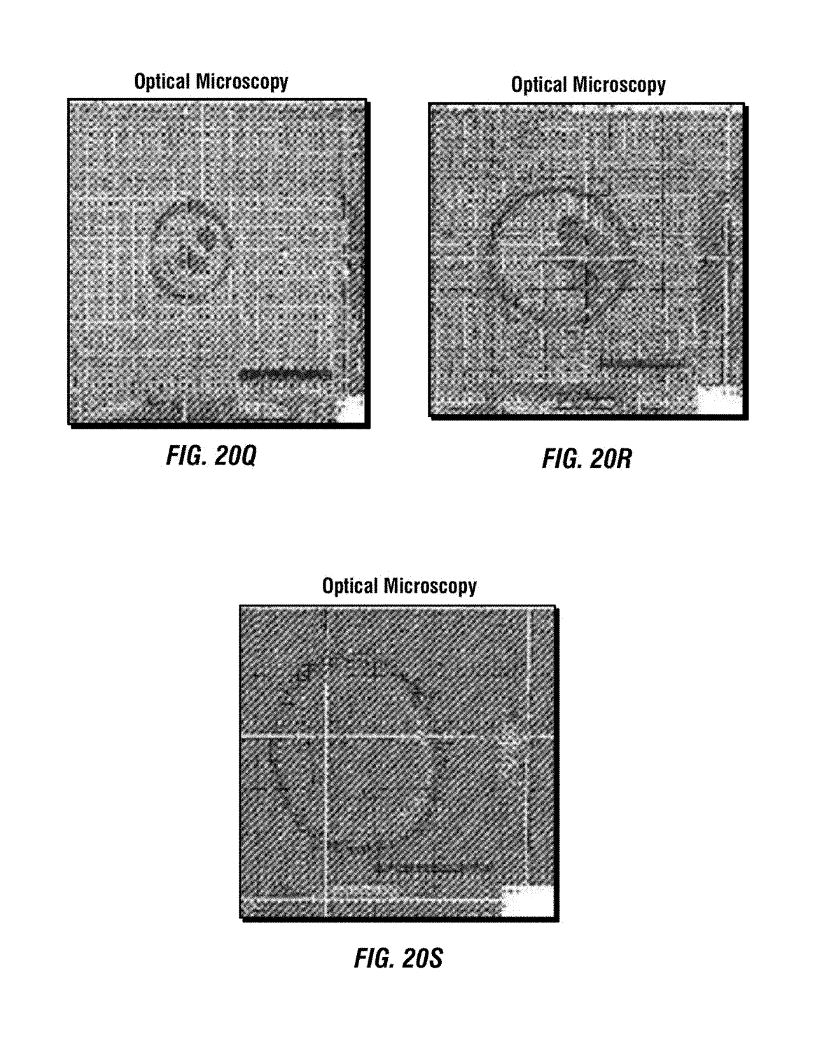

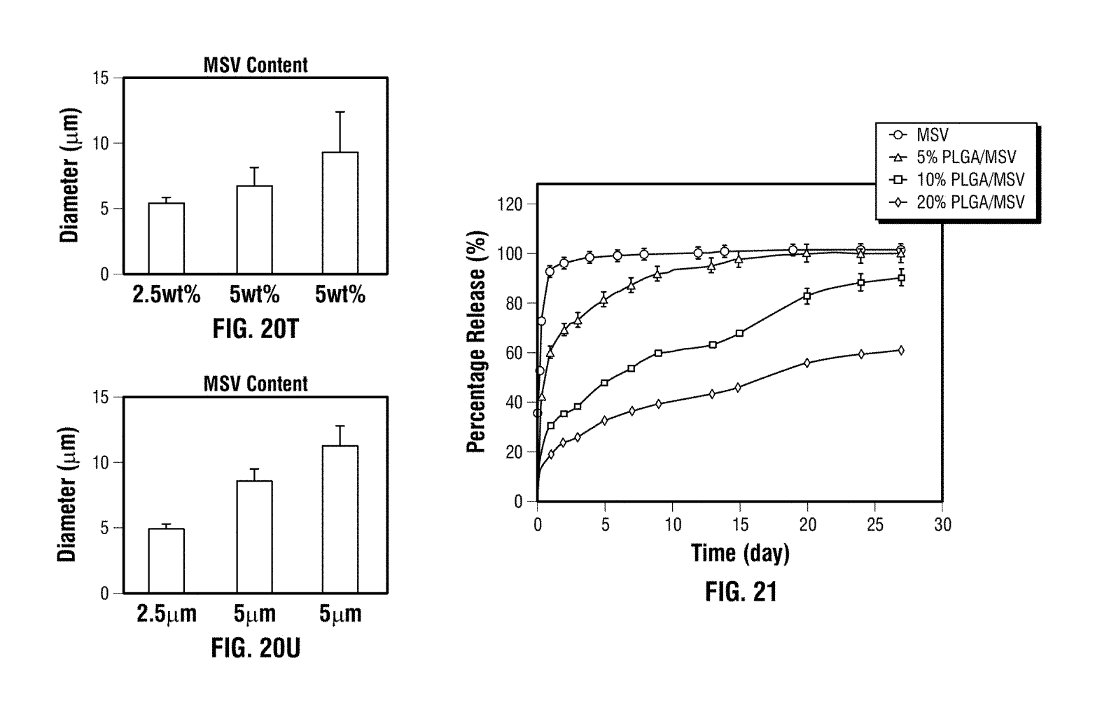

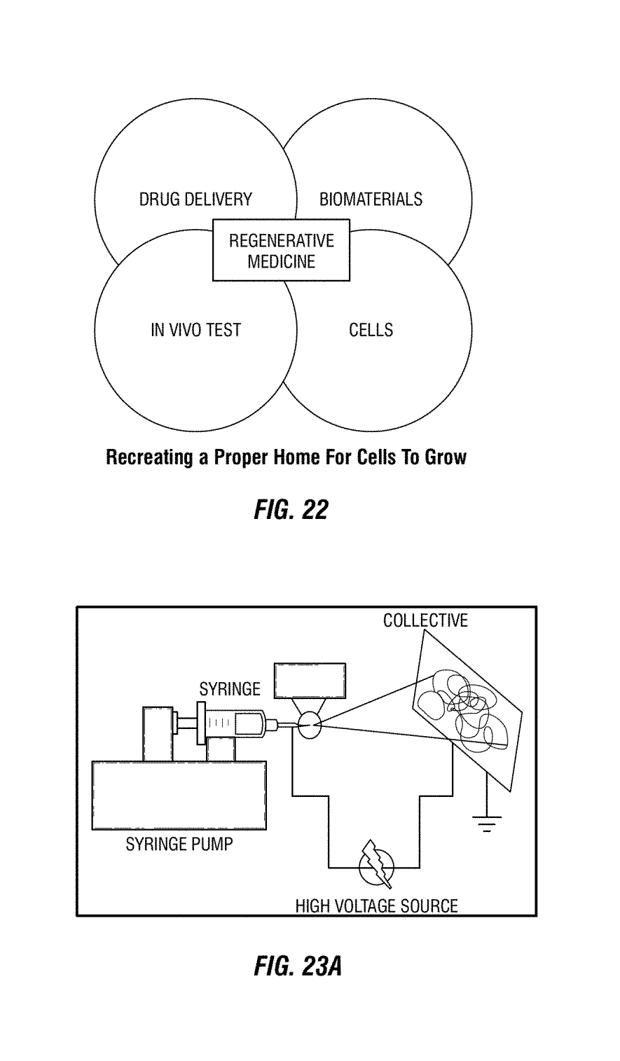

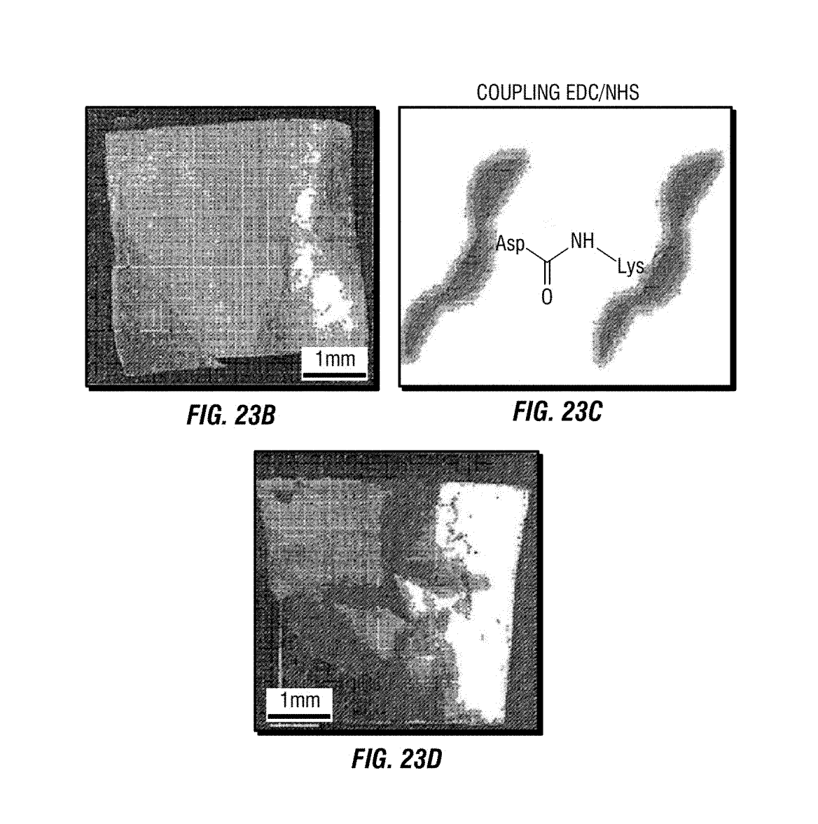

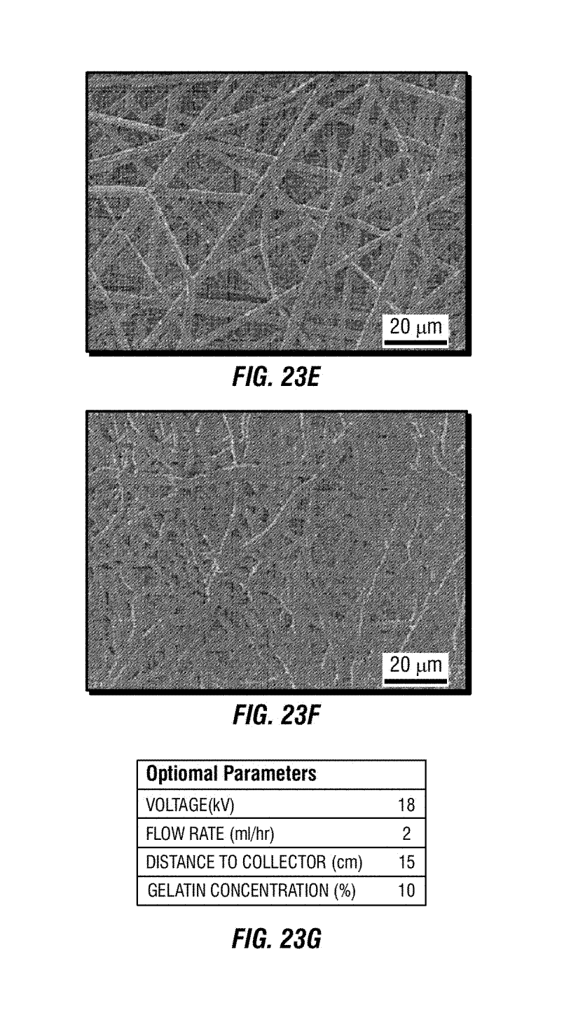

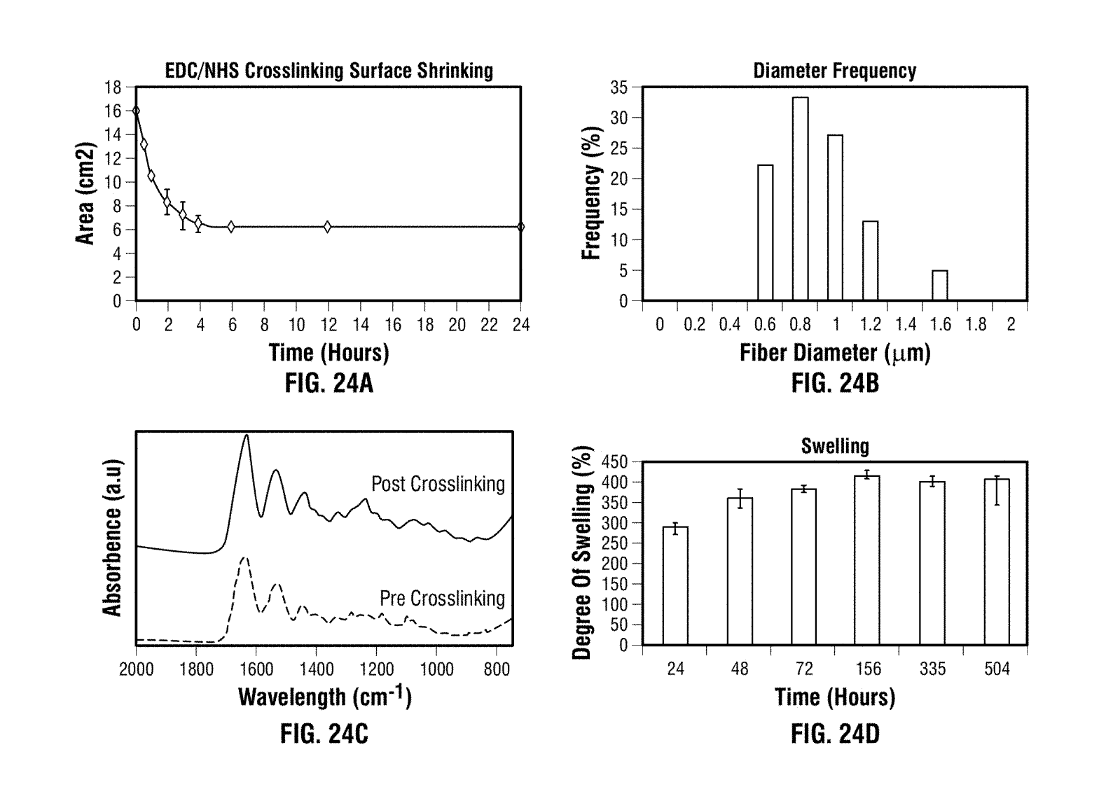

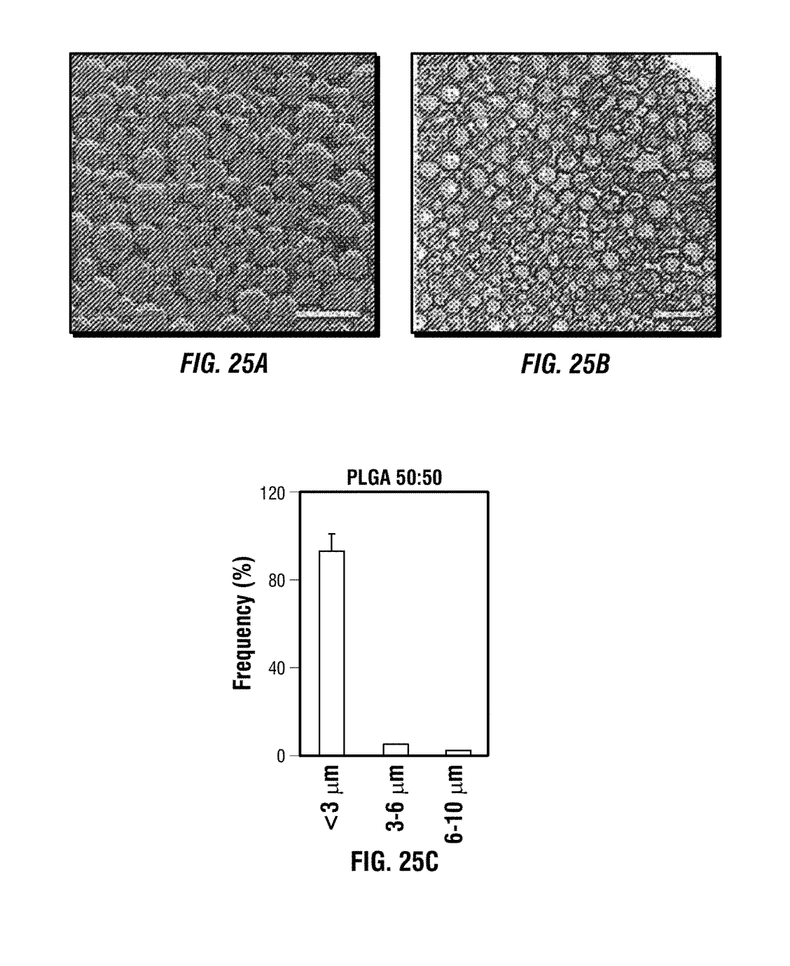

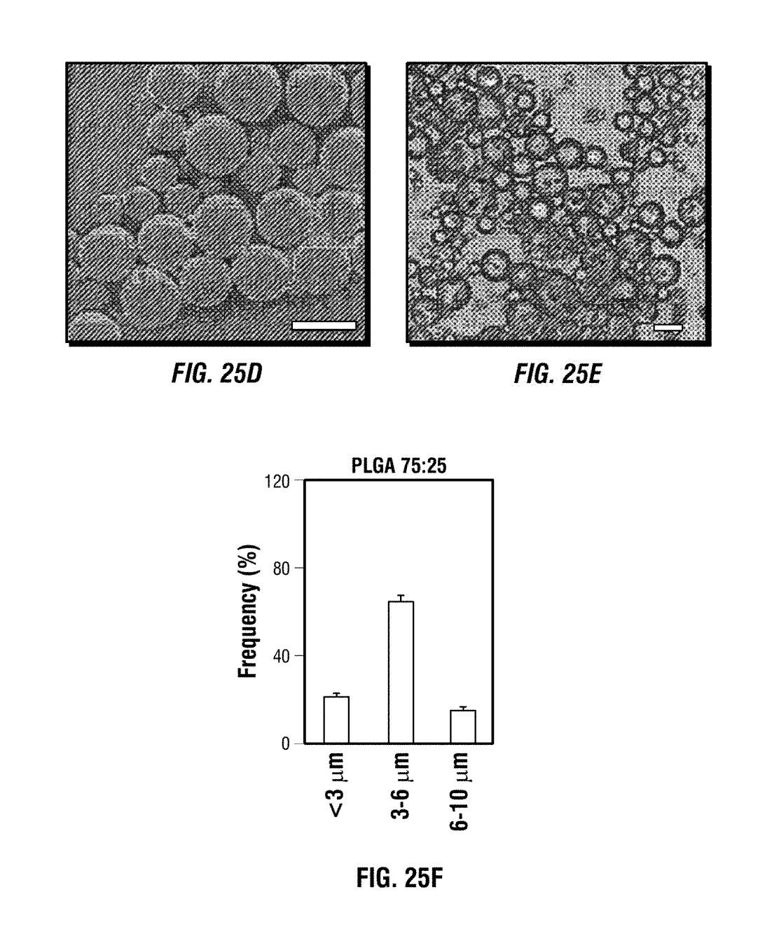

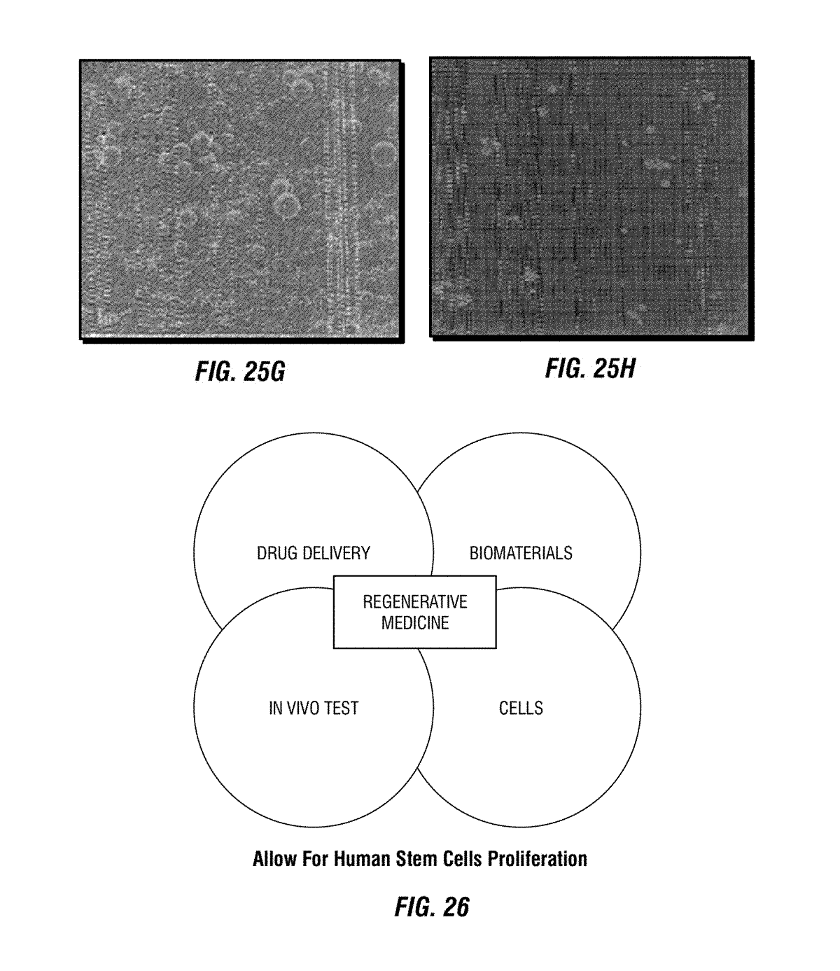

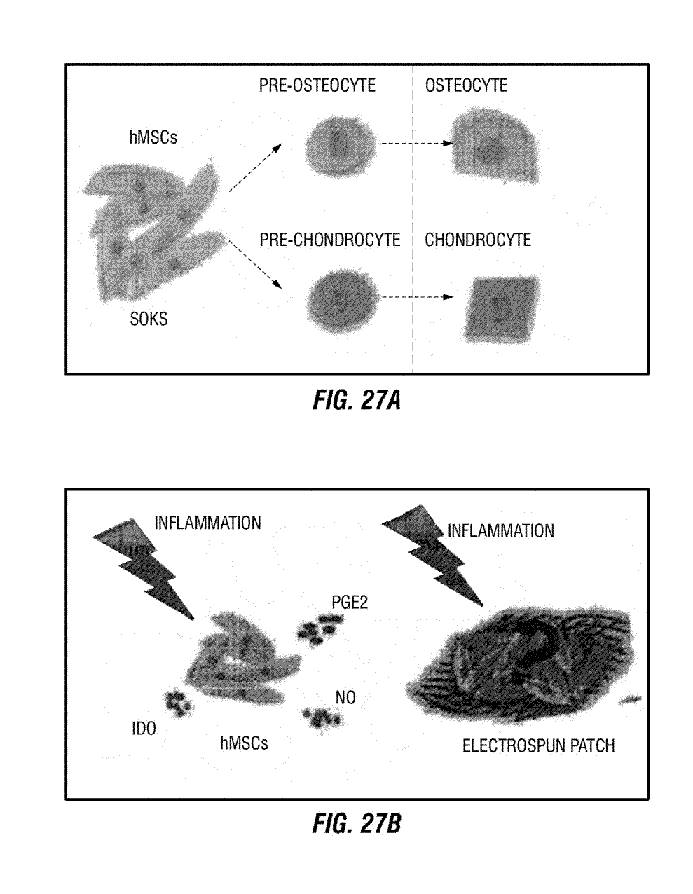

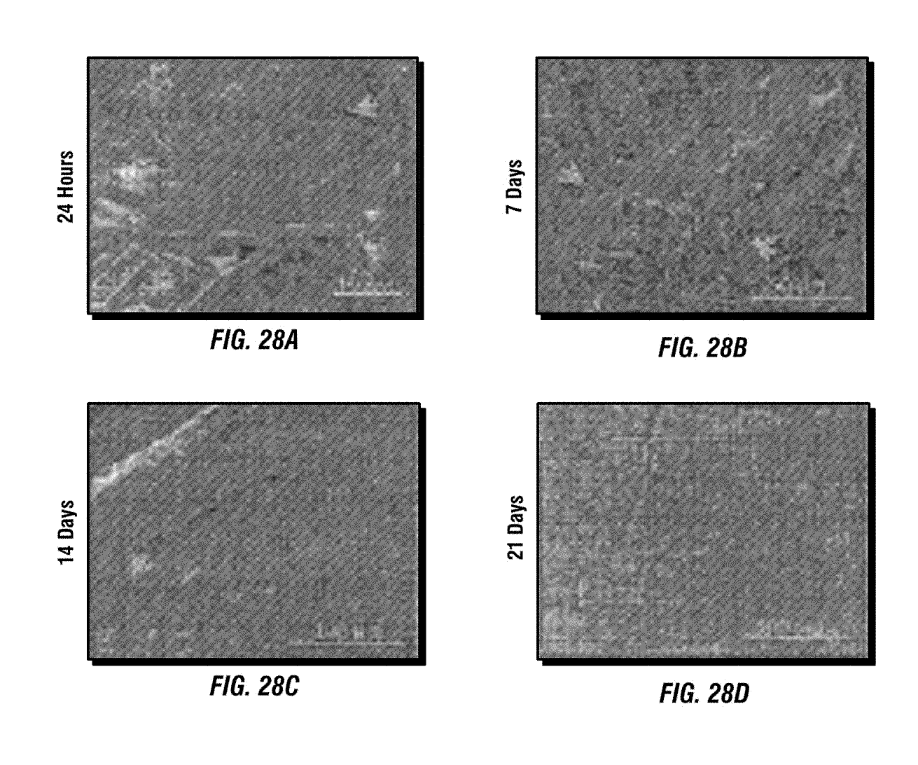

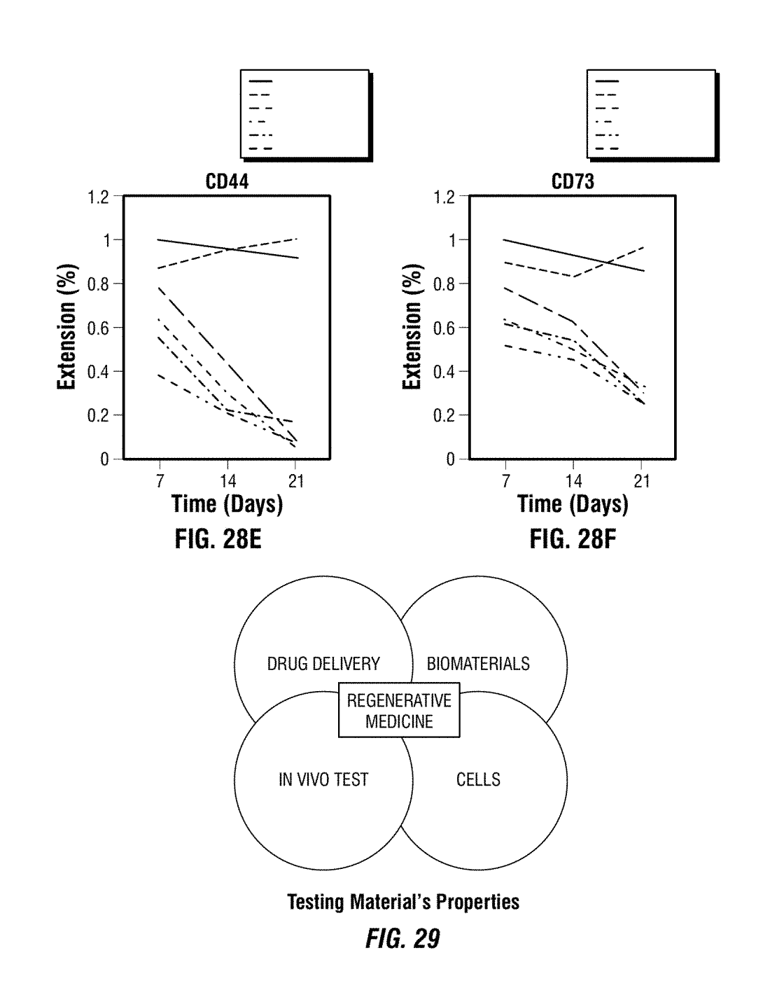

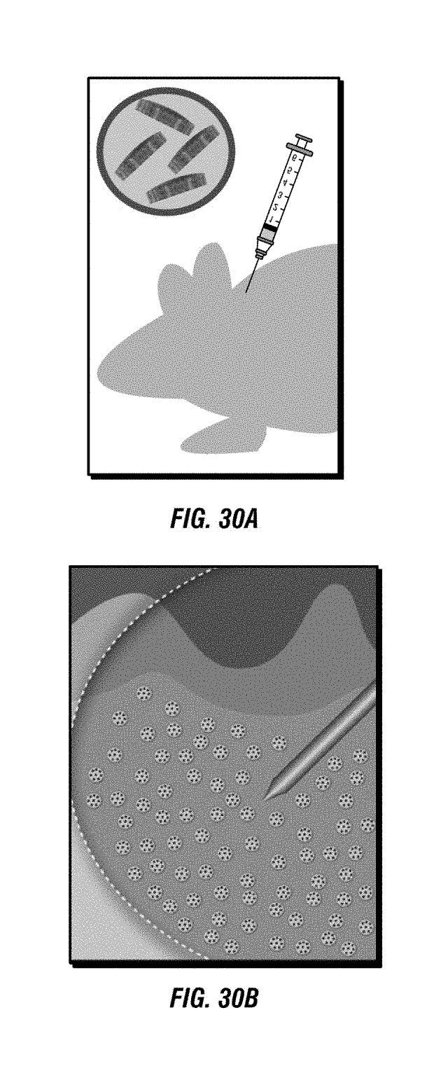

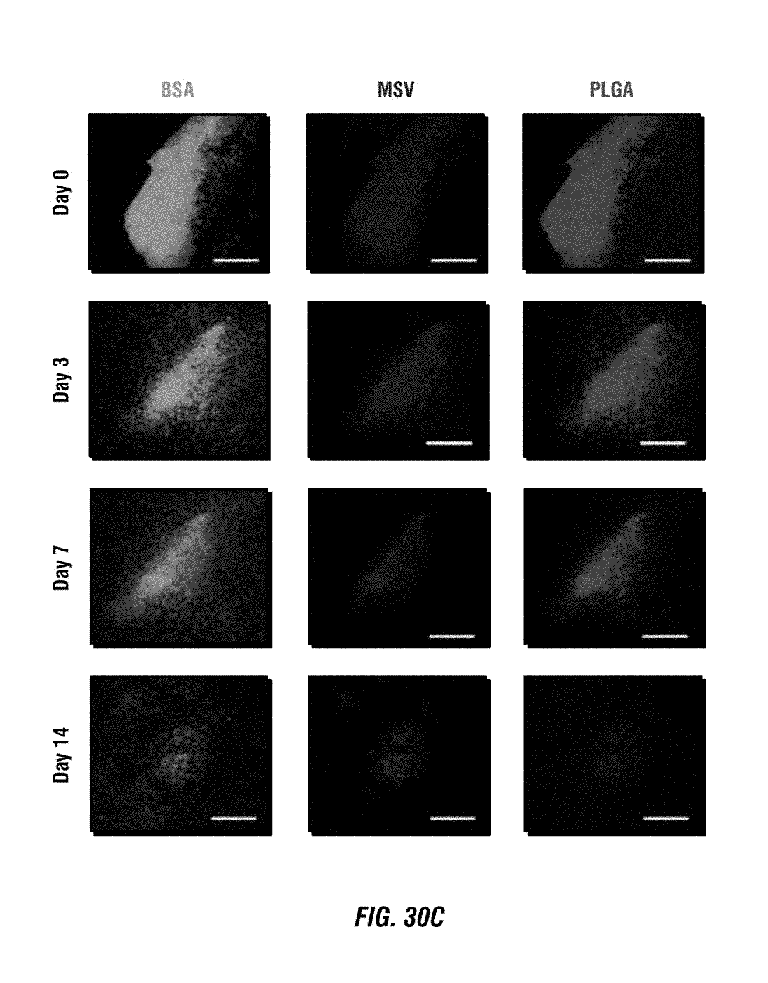

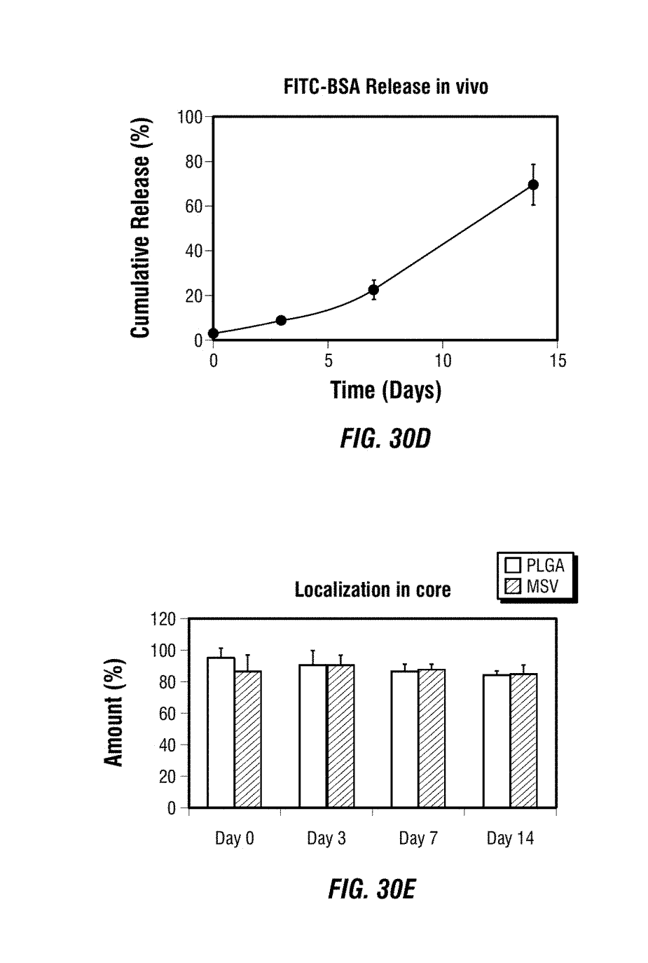

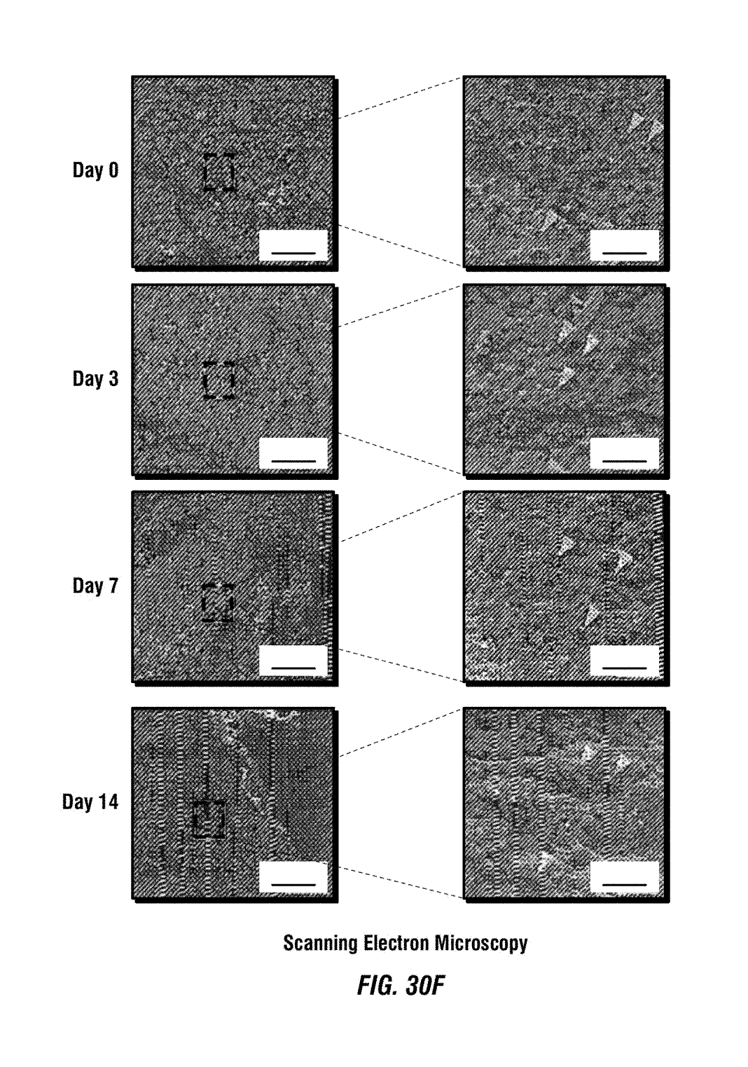

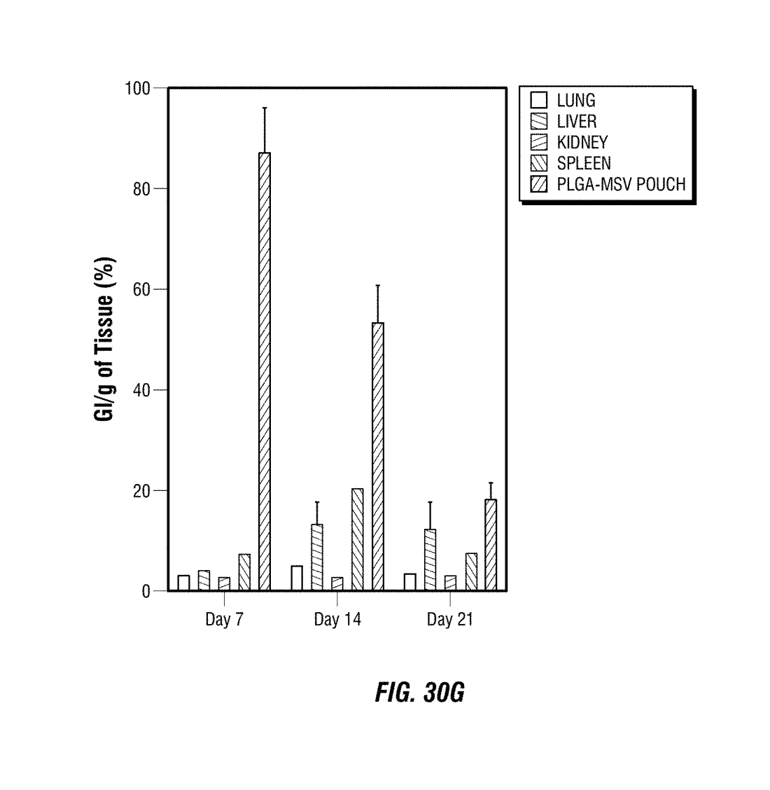

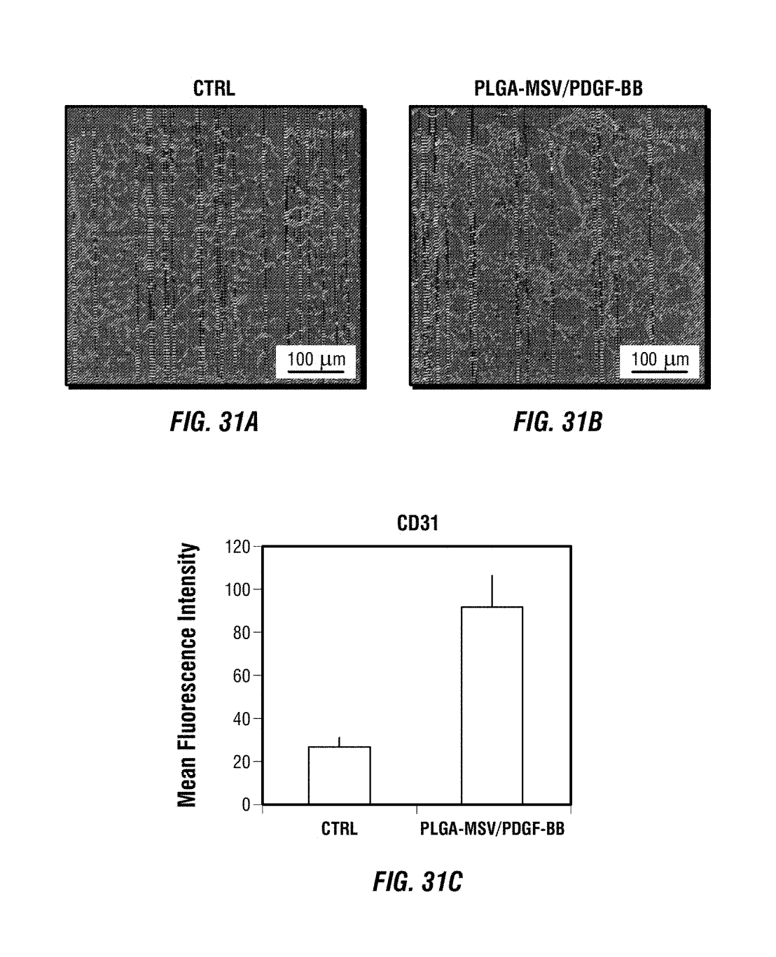

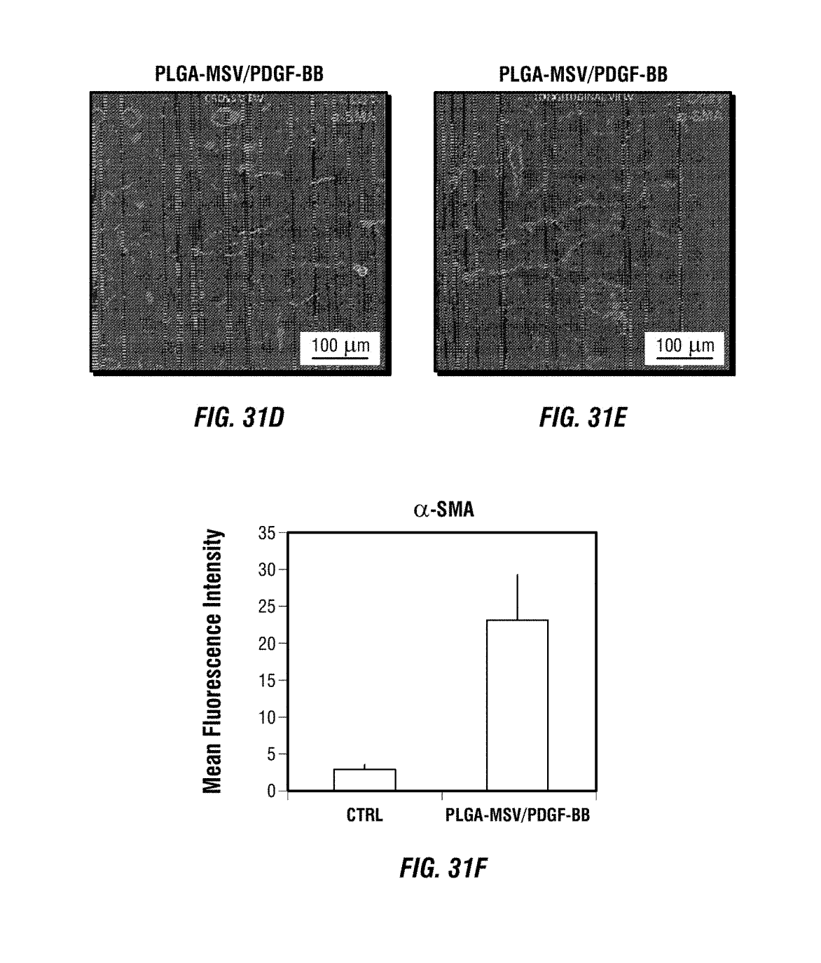

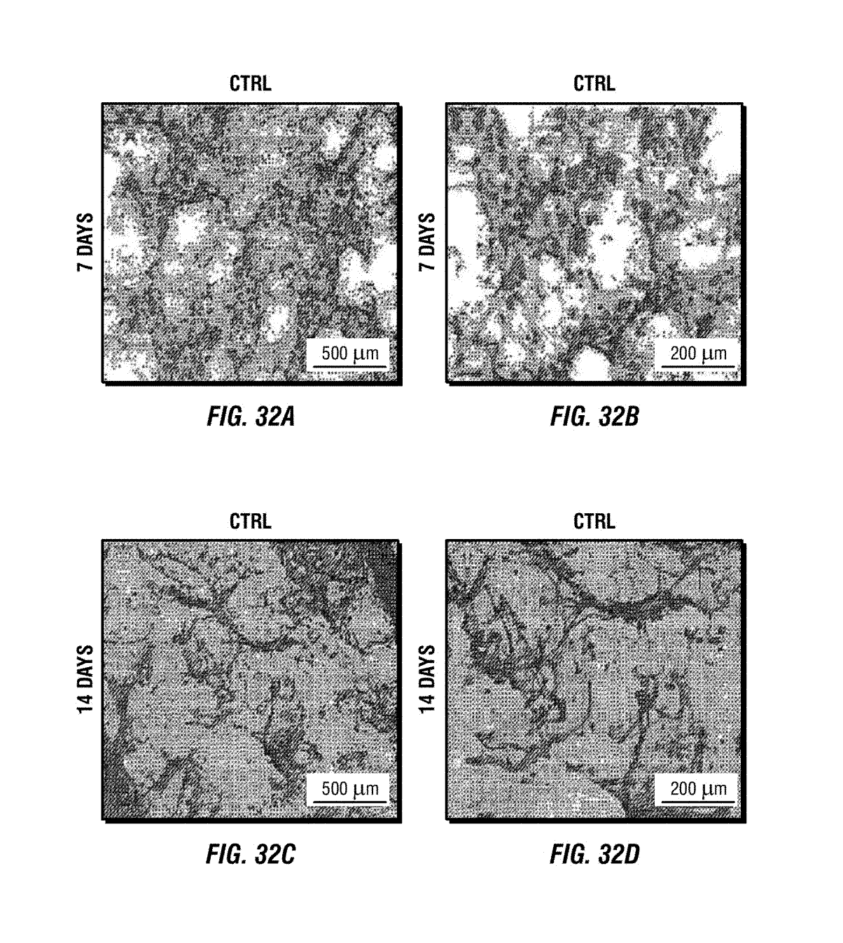

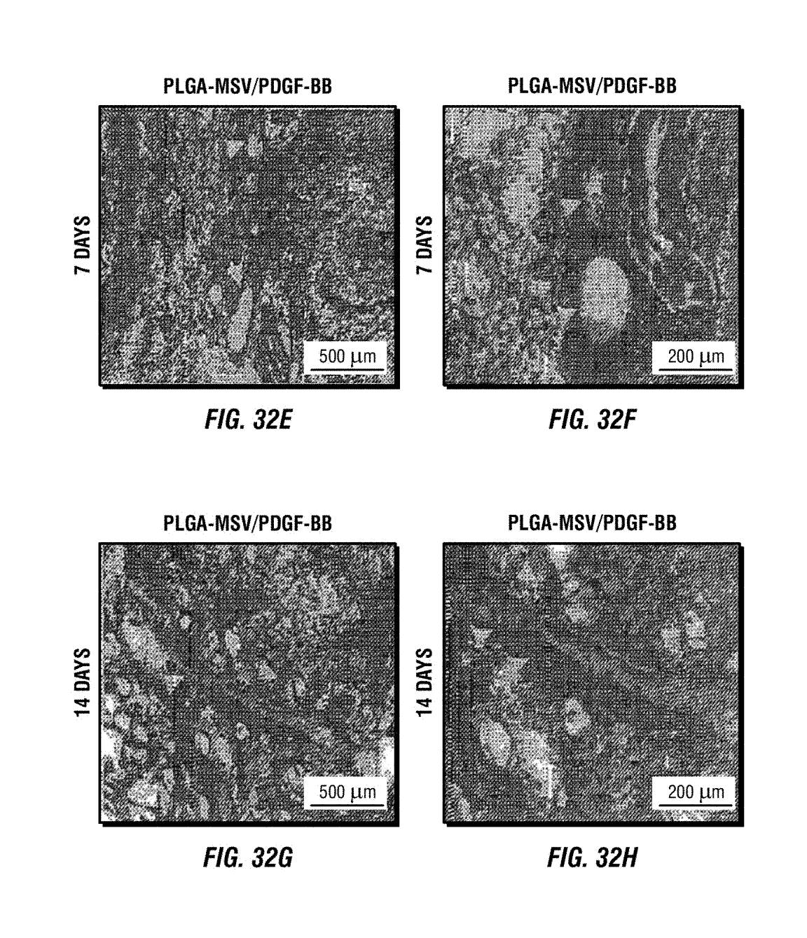

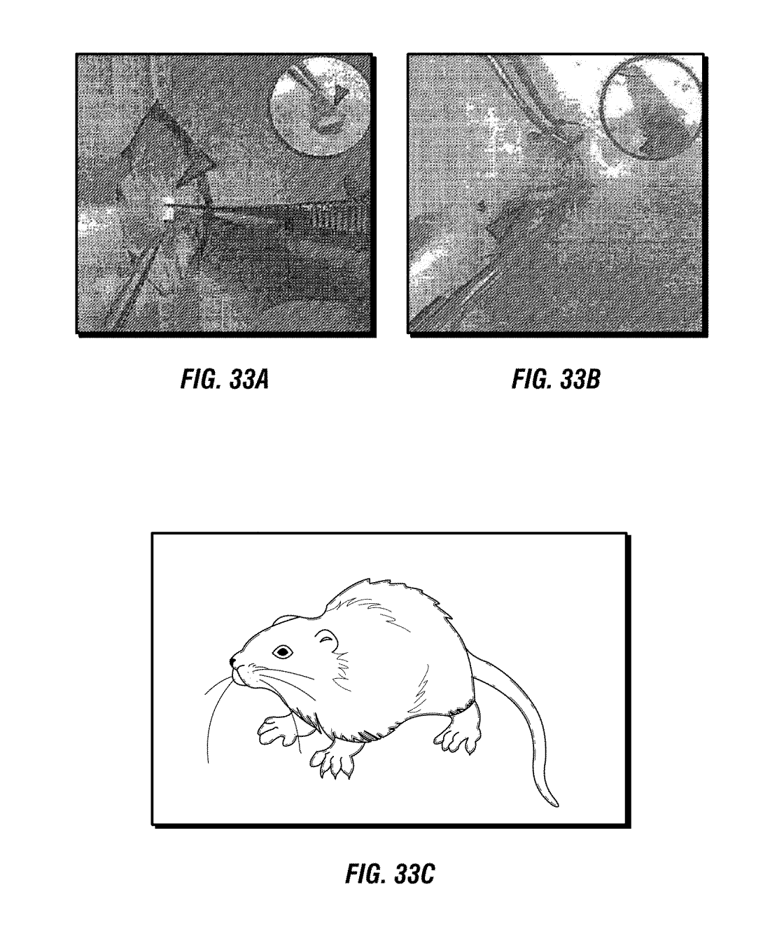

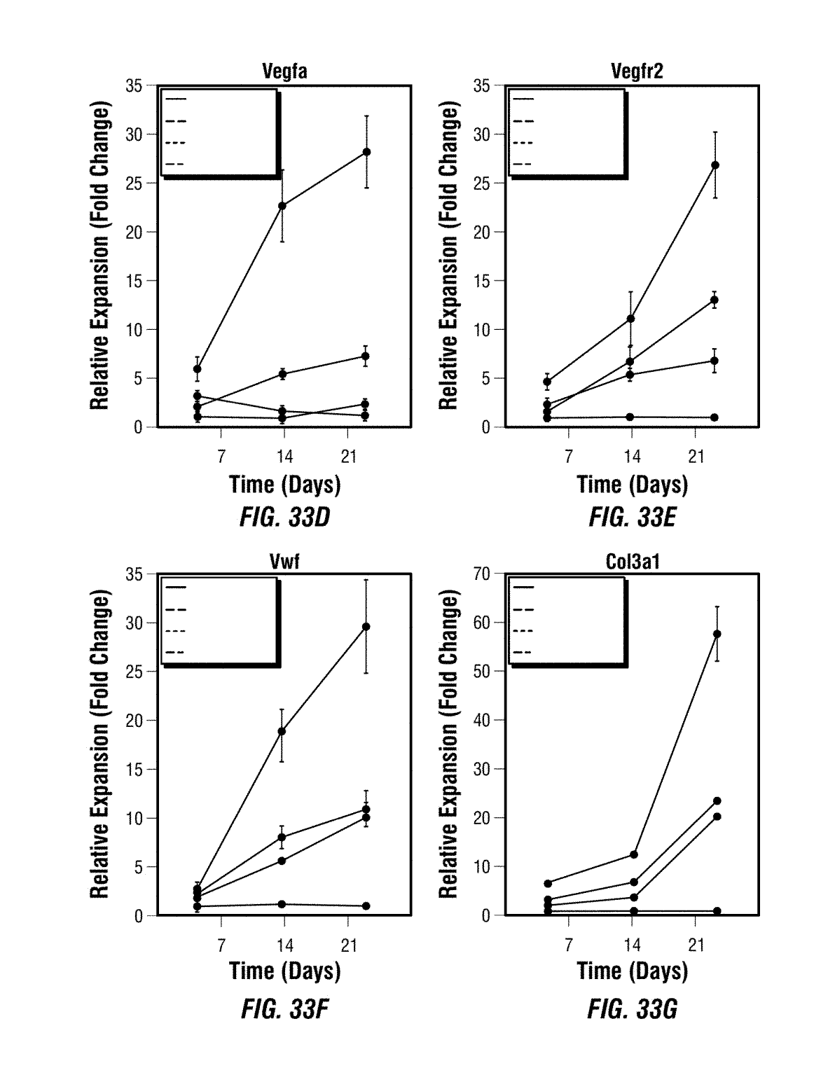

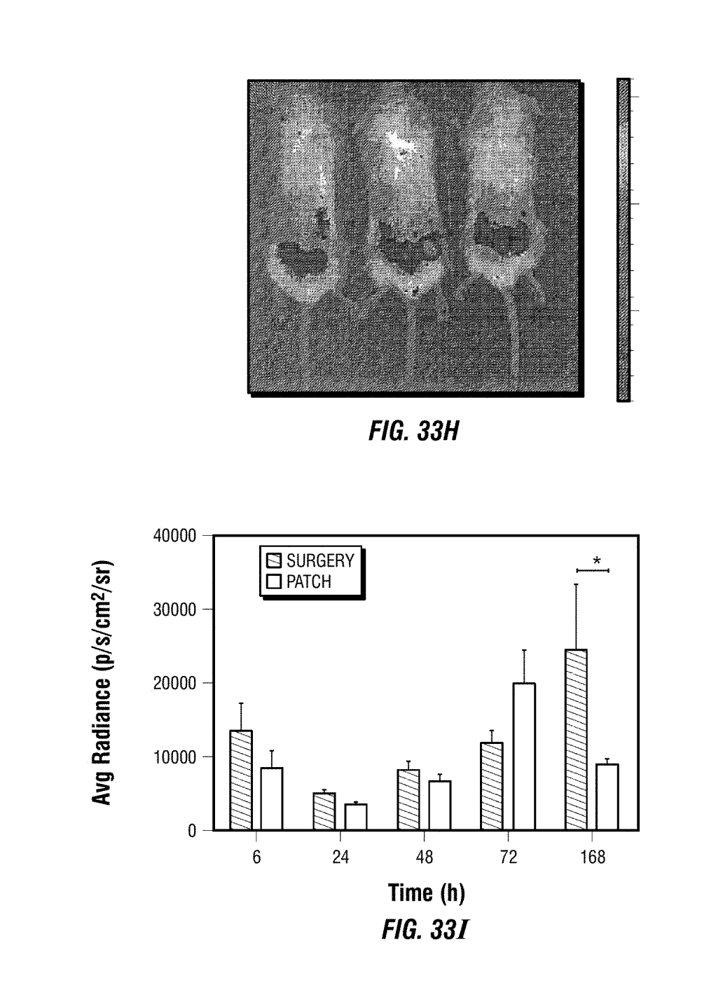

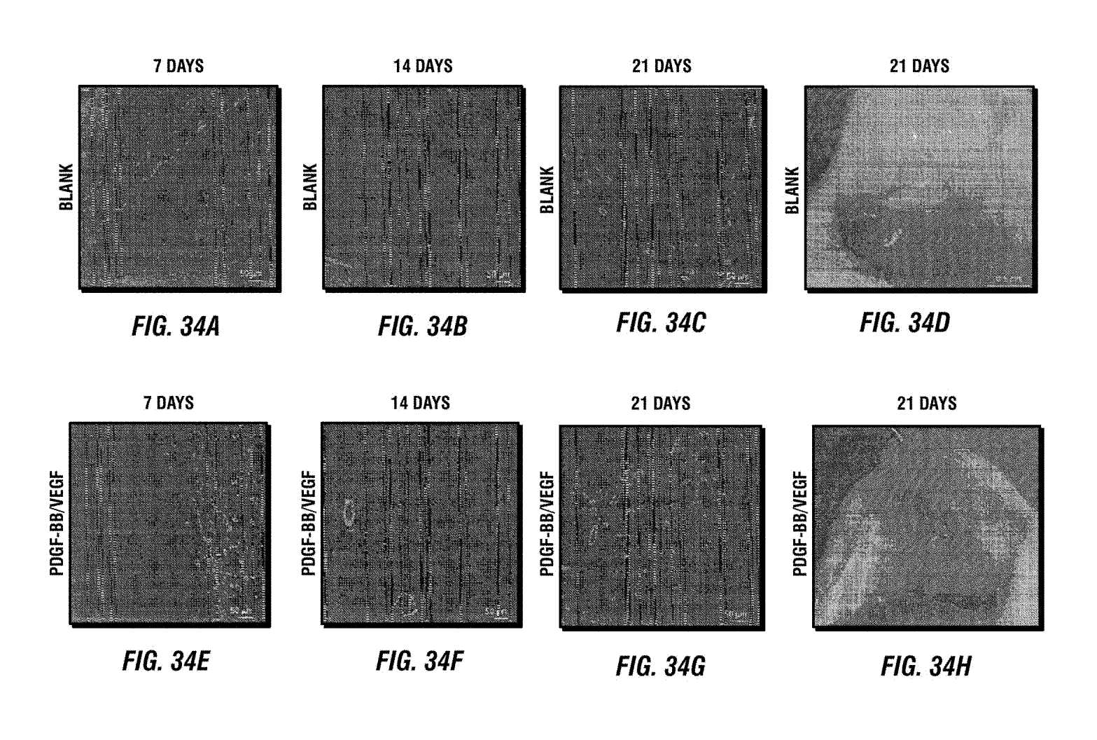

FIG. 16 through FIG. 34H show various embodiments of electrospun patches useful for the controlled release of growth factors to enhance vascularization.

Clinical Benefits

Platforms tailored to release molecules where and when needed;

Biomimetic material to mimic extracellular matrix mechanical and functional properties to promote and direct tissue formation;

Guide proliferation and maintenance of stem cells;

Define an animal model to test a material's properties;

MSV: burst release; (e.g., the higher the copolymer ratio, the more controlled the release);

PLGA coating provides a second level of control over the release;

5% 50:50

VEGF/PLGA-MSV

10% 75:15

PDGF-BB/PLGA-MSV

By integrating PLGA-MSV in the collagen mats (camouflage) it is possible to ensure their spatial confinement and the preservation of their payload's release kinetics resulting in an efficient GFs and molecules release Human MSC were able to proliferate on the patch and maintain their unique features. Human MSCs did not express inflammatory markers, demonstrating that the patch did not elicit any immune response.

Giving cells the right instructions

PLGA-MSV allowed for the fine controlled release of proteins both in vitro and in vivo.

Recreating a proper home for cells to grow

The electrospun patch can be developed using components resembling the ECM composition and can be functionalized with PLGA-MSV.

Allow human stem cells to maintain stemness

hMSC were able to proliferate on the patch and did not express inflammatory markers, demonstrating that the patch did not elicit any immune response.

Determine a material's effect in vivo.

The controlled release of the signaling molecules by the patch resulted in an enhanced local vascularization of each time point and better surgical outcome.

### EXAMPLE 3

### Cardiopatch—Biodegradable, Implantable Patch for Self-directing Autologous Stem Cells to Promote Tissue Regeneration

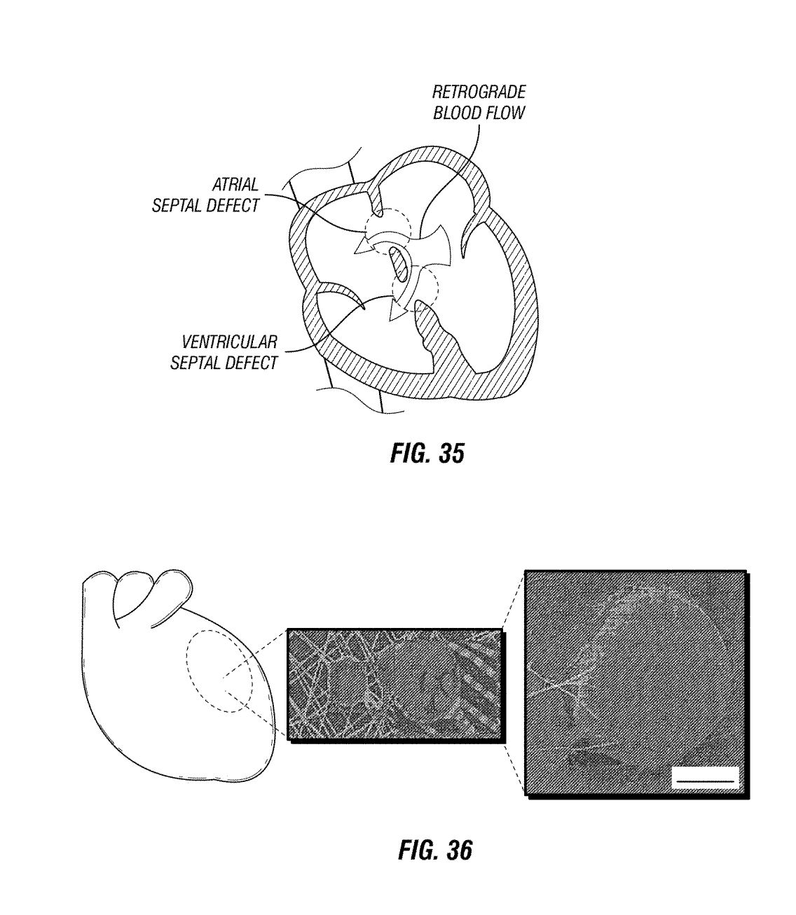

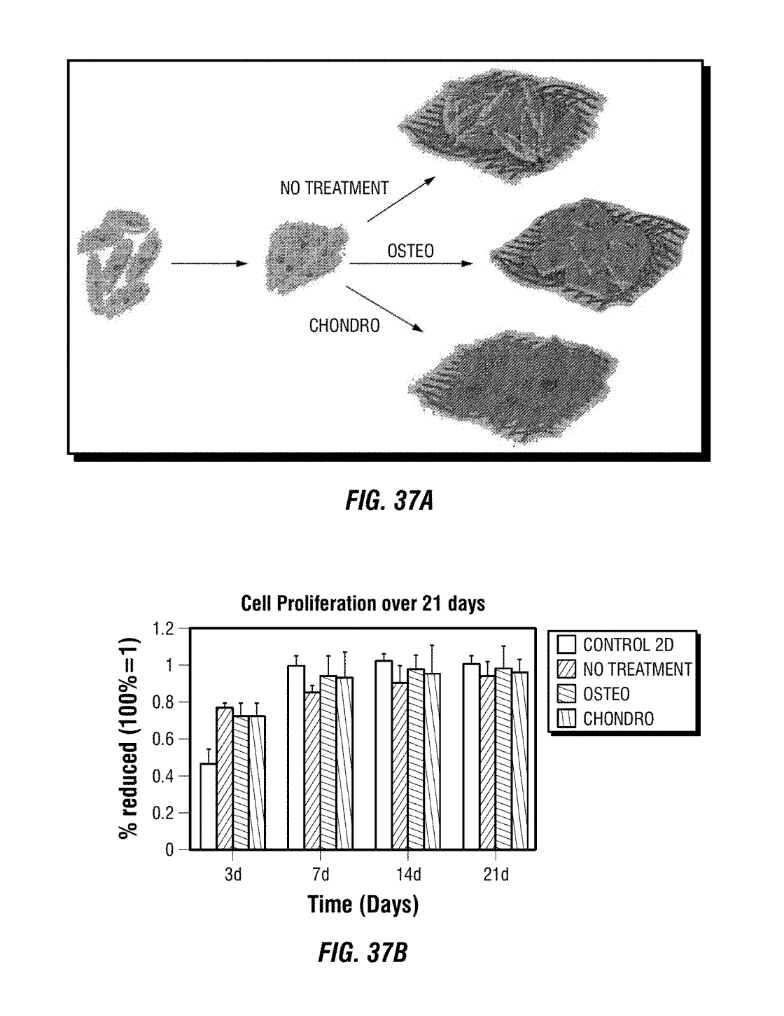

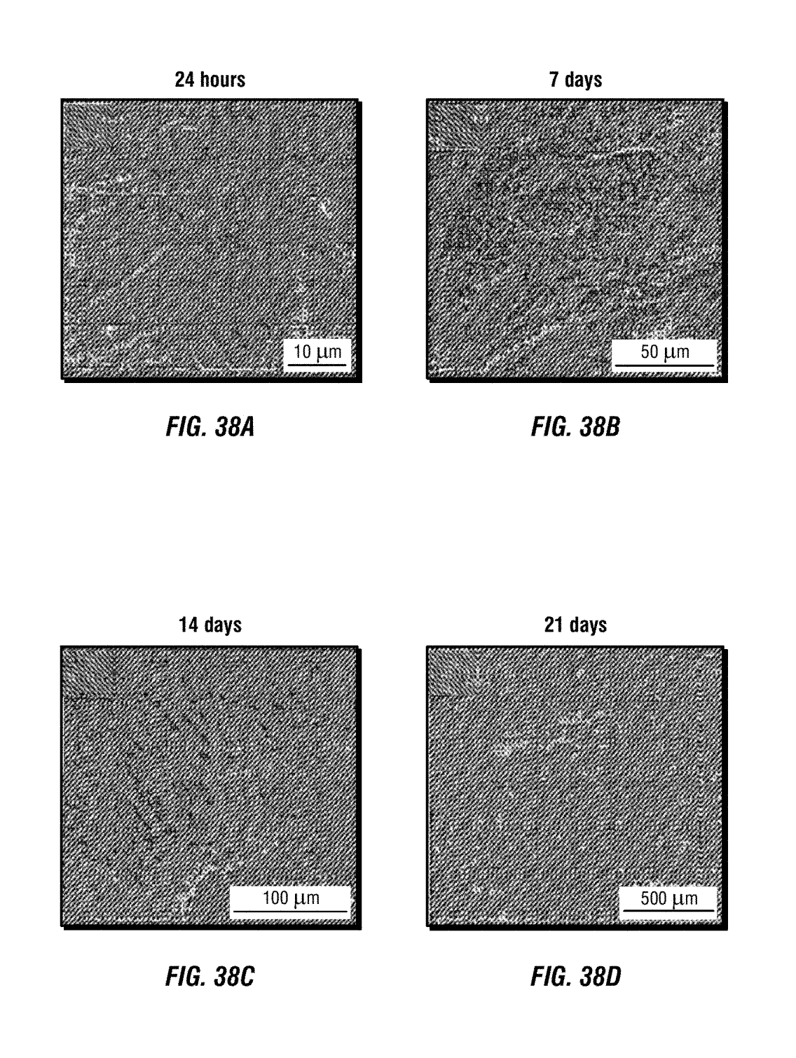

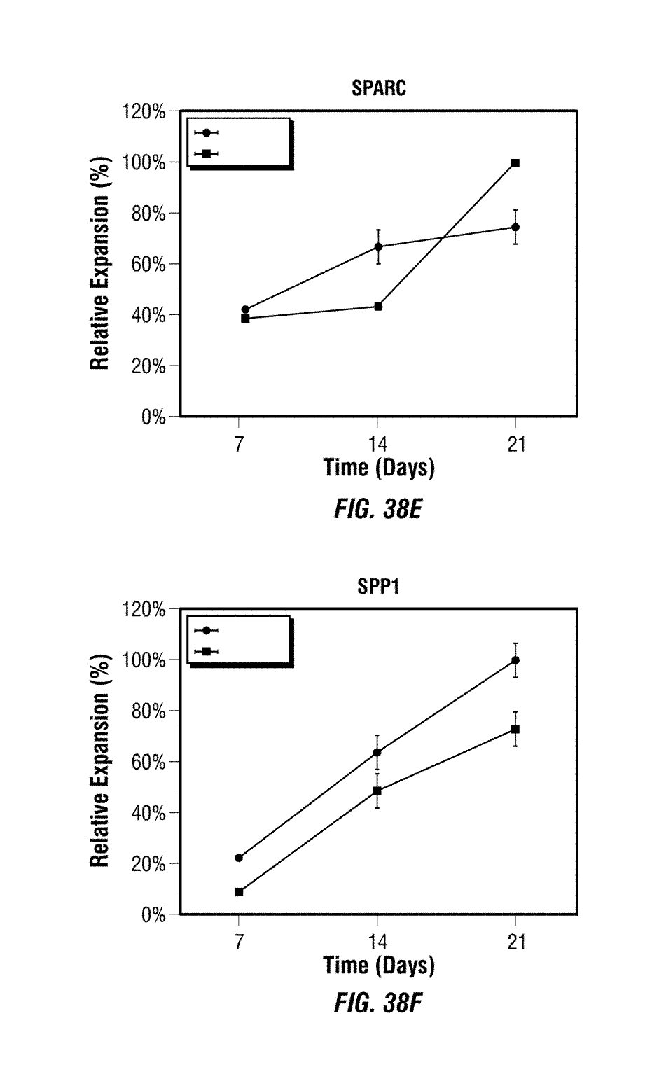

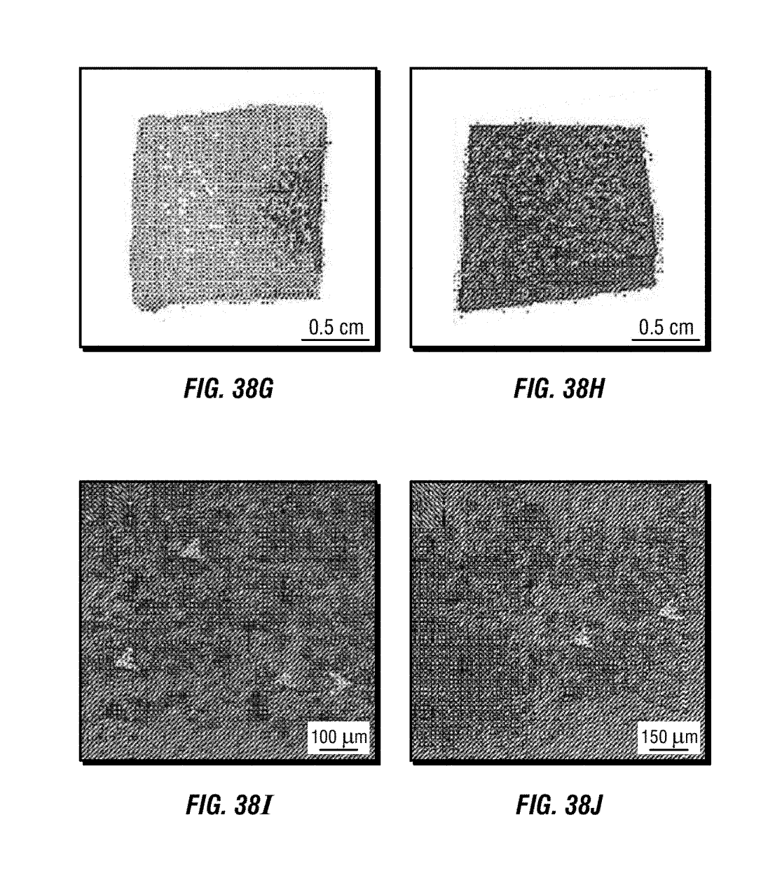

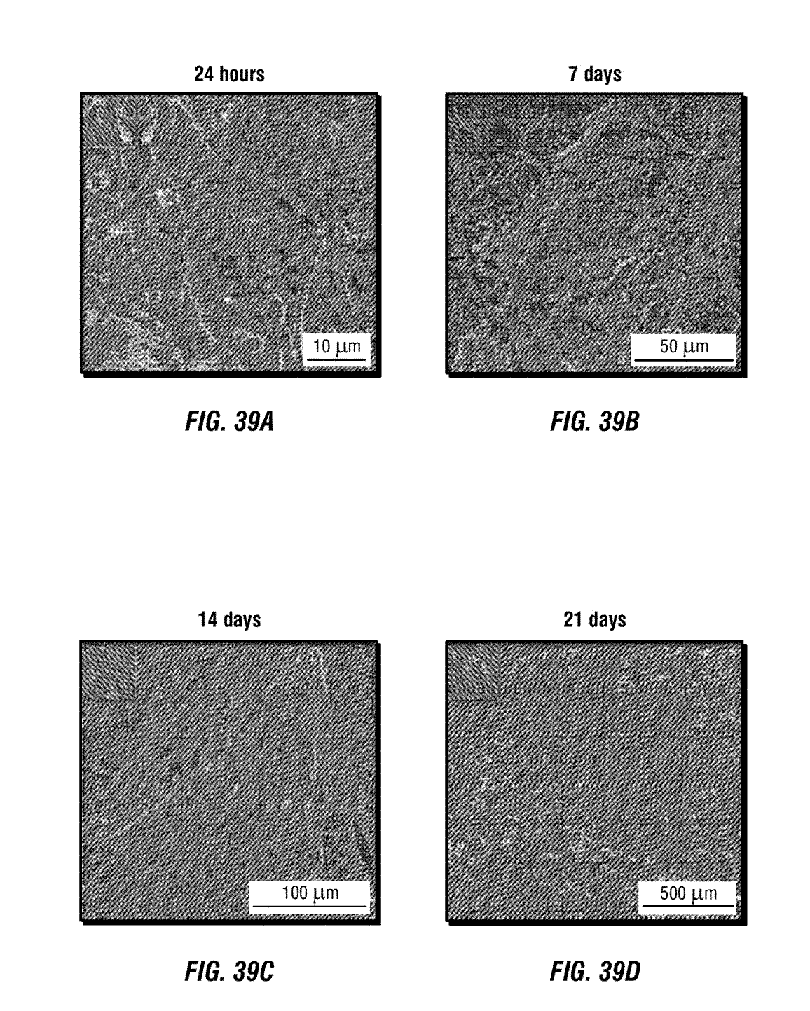

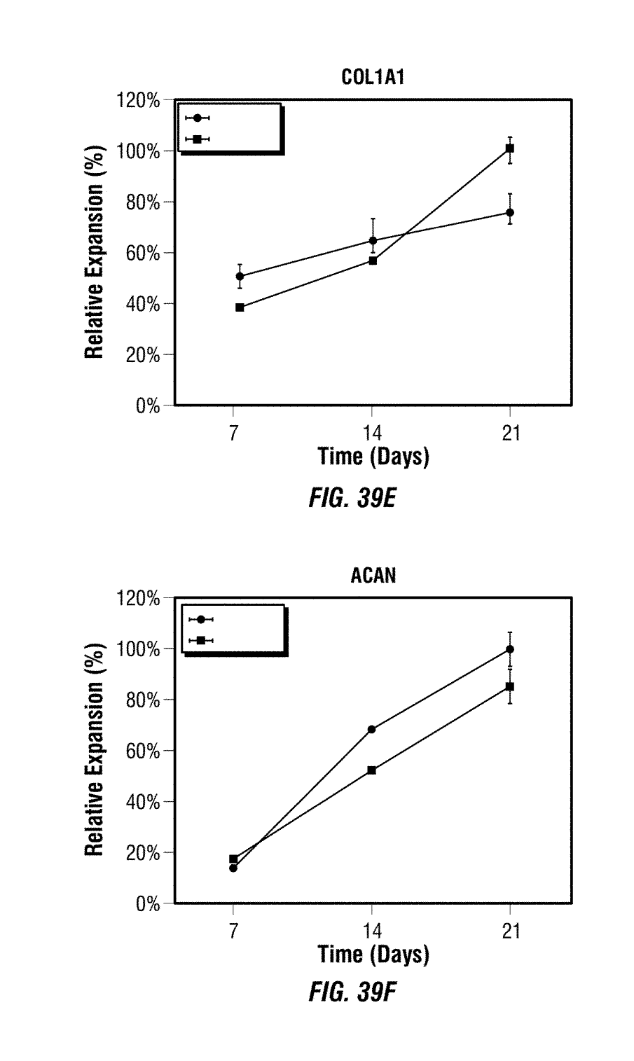

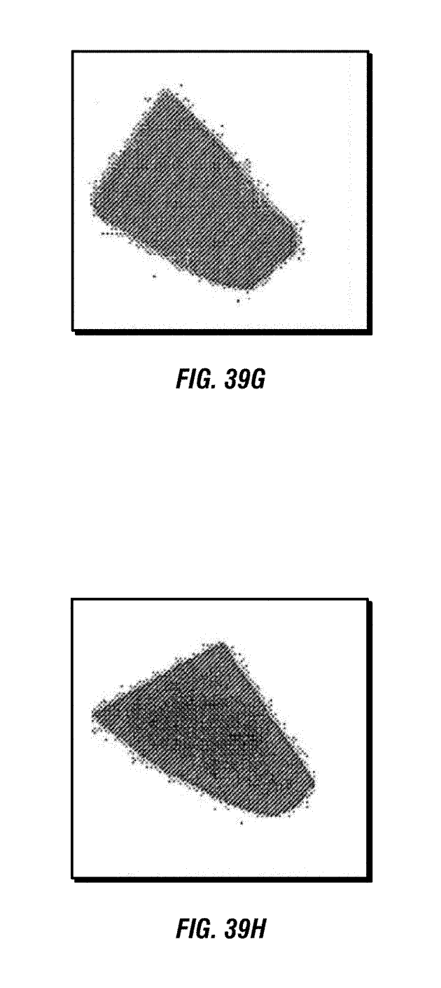

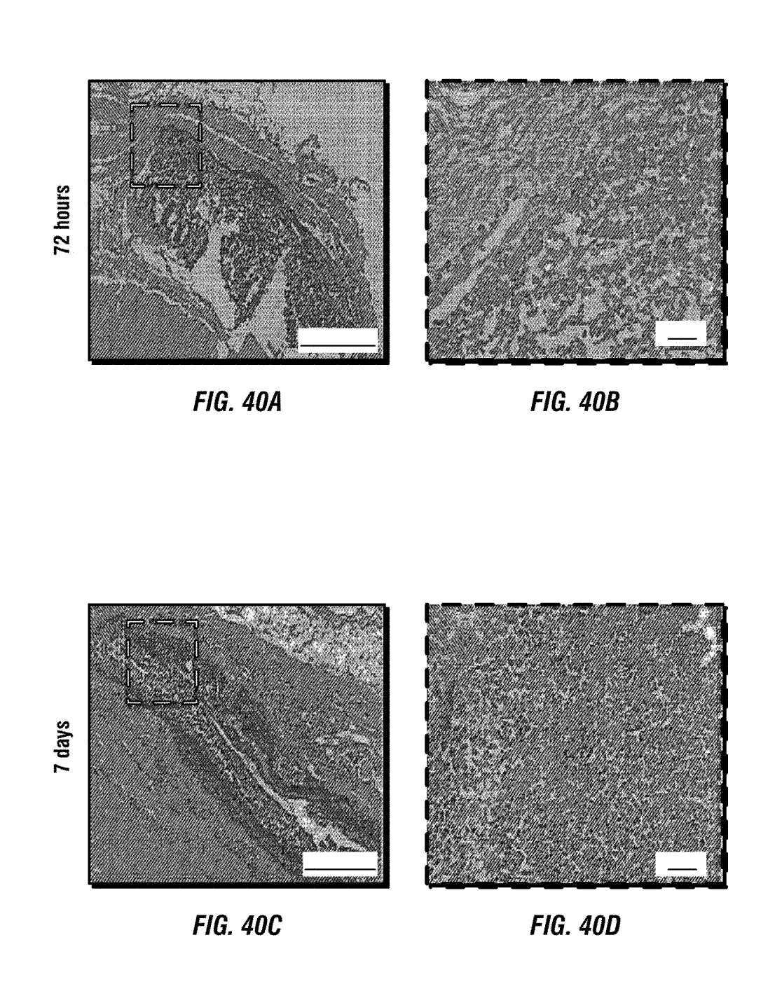

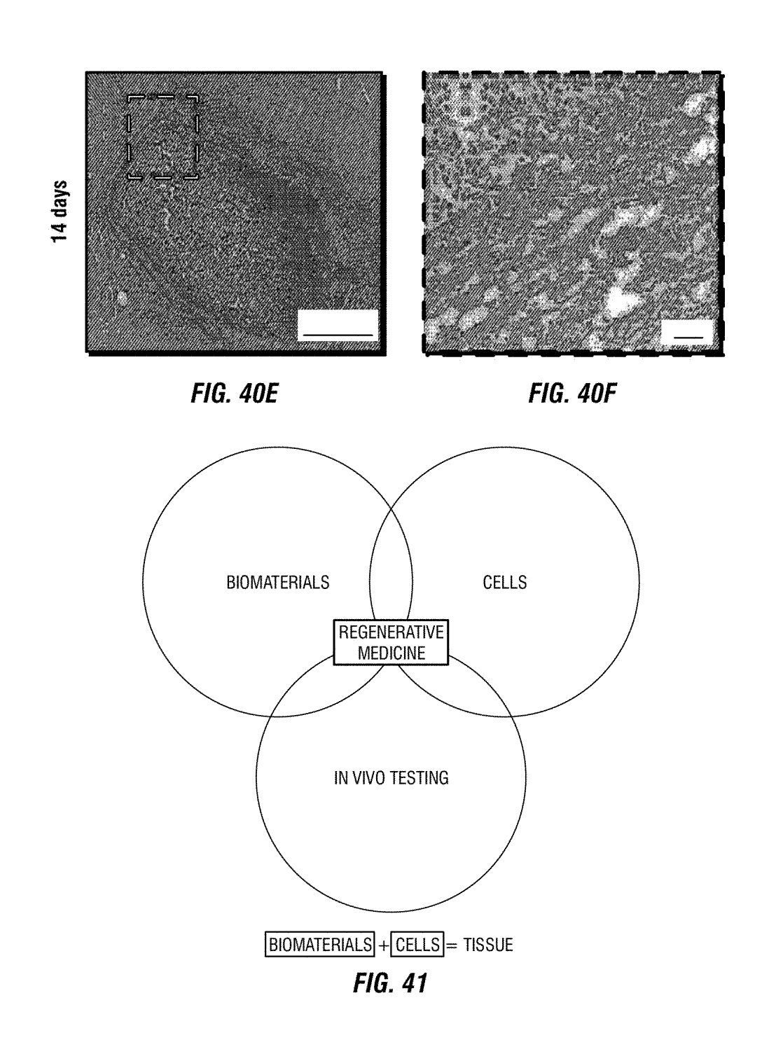

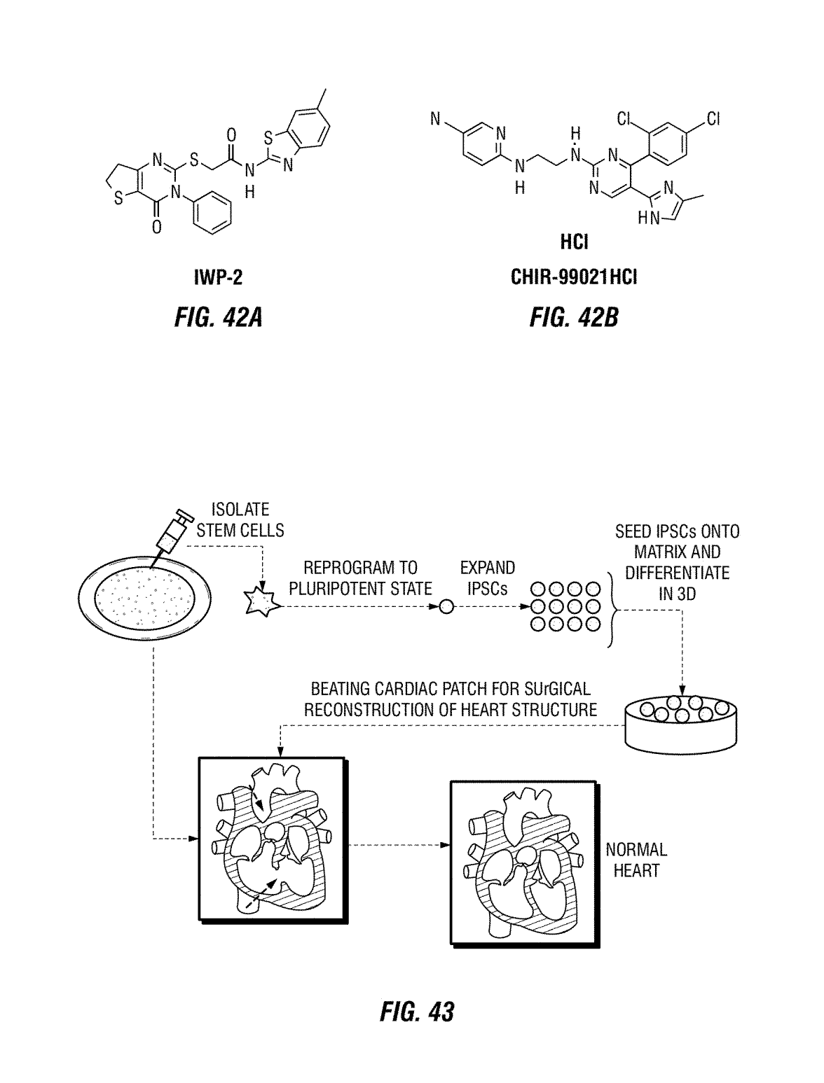

FIG. 35 through FIG. 43 show various views of exemplary biodegradable, implantable electrospun patches (e.g., a “CardioPatch”) useful for treating conditions of the heart, and in delivering self-directing autologous stem cells to promote tissue regeneration.

Epidemiology:

Congenital heart defects (CHD) affect nearly 1% of live births in the United States, equating to nearly 40,000 births per year. Septal wall is the most common and 25% of these will require surgical repair.

Economic impact:

In the US alone an estimated $1.8 billion was the net hospital and care costs in 2011. This represented an average of $23,000 expenditures per patient yearly.

To date, there are no biocompatible cardiac patches (biopatch) capable of growing with the patient, to remodel their structure, and match their function with cardiac tissue.

“Off the Shelf” Materials

Safe, biodegradable, promoting and directing tissue regeneration

A cellularized biopatch capable of self-directing autologous stem cells into a cardiac lineage, which grow with the patient is biodegradable would not only support the native heart structure but also restore function.

Recreating a proper home for cells to grow

The electrospun patch can be developed using components resembling the ECM composition and was fully characterized.

Allow human stem cells to maintain stemness

hMSC were able to proliferate on the patch and maintain their unique features

Determine biocompatibility in vivo

The level of inflammation produced by biopatch is compatible with the inflammation at the site of the surgery or lower.

Temporal modulation of WNT signaling is essential and sufficient for efficient cardiac induction in human pluripotent stem cell (hPSC). Sequential treatment of hPSC with glycogen synthase kinase 3 (GSK-3) inhibitors followed by chemical inhibitors of WNT signaling produce pure functional human cardiomyocytes from hPSC (FIG. 43).

### REFERENCES

The following references, to the extent that they provide exemplary procedural or other details supplementary to those set forth herein, are specifically incorporated herein by reference:

ALTSCHUL, S F et al., “Gapped BLAST and PSI-BLAST: a new generation of protein database search programs,” Nucl. Acids Res., 25(17):3389-3402 (1997).

BADYLAK, S F and GILBERT, T W, “Immune response to biologic scaffold materials,” Semin. Immunol., Elsevier; pp. 109-116 (2008).

BADYLAK, S F et al., “Marrow-derived cells populate scaffolds composed of xenogeneic extracellular matrix,” Exp. Hematol., 29(11):1310-1318 (Nov. 2001).

BADYLAK, S F et al., “Mechanisms by which acellular biologic scaffolds promote functional skeletal muscle restoration,” Biomaterials, 103:128-136 (Oct. 2016).

BEATTIE, A J et al., “Chemoattraction of progenitor cells by remodeling extracellular matrix scaffolds,” Tissue Eng. Part A, 15(5):1119-1125 (May 2009).

BELLON, J et al., “Study of biochemical substrate and role of metalloproteinases in fascia transversalis from hernial processes,” i Eur. J. Clin. Invest., 27:510-516 (XXXX 1997).

BELLOWS, C F et al., “Repair of incisional hernias with biological prosthesis: a systematic review of current evidence, Am. J. Surg., 205:85-101 (2013).

BELLOWS, C F et al., “The effect of bacterial infection on the biomechanical properties of biological mesh in a rat model,” PLoS One, 6:e21228 (2011).

DEVILLE, S et al., “Freeze casting of hydroxyapatite scaffolds for bone tissue engineering,” Biomaterials, 27(32):5480-5489 (Nov. 2006).

EYRE-BROOK, A L “The periosteum: its function reassessed,” Clin. Orthop. Relat. Res., 189:300-307 (Oct. 1984).

FILARDO, G et al., “New bio-ceramization processes applied to vegetable hierarchical structures for bone regeneration: an experimental model in sheep. Tissue Engineering Part A, 20(3-4):763-73 (2013).

FREYTES, D O et al., “Uniaxial and biaxial properties of terminally sterilized porcine urinary bladder matrix scaffolds,” J. Biomed. Mater. Res. B Appl. Biomater., 84(2):408-414 (Feb. 2008).

GILBERT, T W et al., “Degradation and remodeling of small intestinal submucosa in canine Achilles tendon repair,” J. Bone Joint Surg. Am., 89(3):621-630 (Mar. 2007).

GRIBSKOV, M, and BURGESS, R R, “Sigma factors from E. coli, B. subtilis, phage SP01, and phage T4 are homologous proteins,” Nucleic Acids Res., 14(16):6745-6763 (Aug. 1986).

GUGALA, Z et al., (Eds.) New Approaches in the Treatment of Critical-Size Segmental Defects in Long Bones. Macromolecular Symposia; Wiley Online Library (2007).

HALE, W G, and MARGHAM, J P, “HARPER COLLINS DICTIONARY OF BIOLOGY,” HarperPerennial, New York (1991).

HALL, M J et al., “National hospital discharge survey: 2007 summary,” Natl Health Stat Report, 29:1-20 (2010).

HARDMAN, J G, and LIMBIRD, L E, (Eds.), “GOODMAN AND GILMAN'S THE PHARMACOLOGICAL BASIS OF THERAPEUTICS” 10^{th} Edition, McGraw-Hill, New York (2001).

HODDE, J et al., “Effects of sterilization on an extracellular matrix scaffold: part II. Bioactivity and matrix interaction,” J. Mater. Sci. Mater. Med., 18(4):545-550 (Apr. 2007).

HOFFMAN, M D and BENOIT, D S, “Emerging ideas: engineering the periosteum: revitalizing allografts by mimicking autograft healing,” Clin. Orthopaed. Rel. Res., 471(3):721-726 (2013).

KANG, Y et al., “Engineering vascularized bone grafts by integrating a biomimetic periosteum and β-TCP scaffold,” ACS Appl. Mat. Interface, 6(12):9622-9633 (2014).

KING, K F “Periosteal pedicle grafting in dogs,” J. Bone Joint Surg. Br., 58(1):117-121 (Feb. 1976).

KON, E et al., “A novel nano-composite multi-layered biomaterial for treatment of osteochondral lesions: technique note and an early stability pilot clinical trial,” Injury, 41(7):693-701 (Jul. 2010).

KON, E et al., “Novel nano-composite multi-layered biomaterial for the treatment of multifocal degenerative cartilage lesions,” Knee Surg. Sports Traumatol. Arthroscop., 17(11): 1312-1315 (Nov. 2009).

KON, E et al., “Novel nano-composite multilayered biomaterial for osteochondral regeneration a pilot clinical trial,” Am. J. Sports Med., 39(6):1180-1190 (Jun. 2011).

KON, E et al., “Novel nanostructured scaffold for osteochondral regeneration: pilot study in horses,” J. Tissue Eng. Regen. Med., 4(4):300-308 (Jun. 2010).

KON, E et al., “Orderly osteochondral regeneration in a sheep model using a novel nano-composite multilayered biomaterial,” J. Orthop. Res., 28(1): 116-124 (Jan. 2010).

KON, E et al., “Platelet autologous growth factors decrease the osteochondral regeneration capability of a collagen-hydroxyapatite scaffold in a sheep model,” BMC Musculoskelet. Disord., 11:220 (Sept. 2010).

KOSTOPOULOS, L and KARRING, T, “Role of periosteum in the formation of jaw bone: An experiment in the rat,” J. Clin. Periodontal., 22(3):247-254 (Mar. 1995).

KYTE, J, and DOOLITTLE, R F, “A simple method for displaying the hydropathic character of a protein,” J. Mol. Biol., 157(1): 105-132 (1982).

LONG, T et al., “The effect of mesenchymal stem cell sheets on structural allograft healing of critical sized femoral defects in mice,” Biomaterials, 35(9):2752-2759 (2014).

MARCACCI, M et al., “Stem cells associated with macroporous bioceramics for long bone repair: 6- to 7-year outcome of a pilot clinical study,” Tissue Eng., 13(5):947-955 (May 2007).

MINARDI, S et al., “Evaluation of the osteoinductive potential of a bio-inspired scaffold mimicking the osteogenic niche for bone augmentation,” Biomaterials, 62:128-137 (Sept. 2015).

MINARDI, S et al., “Multiscale patterning of a biomimetic scaffold integrated with composite microspheres,” Small, 10(19):3943-3953 (Oct. 2014).

MURPHY, M B et al., “Adult and umbilical cord blood-derived platelet-rich plasma for mesenchymal stem cell proliferation, chemotaxis, and cryo-preservation,” Biomaterials, 33(21):5308-5316 (2012).

MURPHY, M B et al., “Engineering a better way to heal broken bones,” Chem. Eng. Prog., 106(11):37-43 (XXXX 2010).

MURPHY, M B et al., “Multi-composite bioactive osteogenic sponges featuring mesenchymal stem cells, platelet-rich plasma, nanoporous silicon enclosures, and peptide amphiphiles for rapid bone regeneration,” J. Funct. Biomat., 2(2):39-66 (2011).

NEEDLEMAN, S B and WUNSCH, C D, “A general method applicable to the search for similarities in the amino acid sequence of two proteins,” J. Mol. Biol., 48(3):443-453 (1970).

NIEPONICE, A et al., “Reinforcement of esophageal anastomoses with an extracellular matrix scaffold in a canine model,” Ann. Thorac. Surg., 82(6):2050-2058 (Dec. 2006).

NOAH, E M et al., “Impact of sterilization on the porous design and cell behavior in collagen sponges prepared for tissue engineering,” Biomaterials, 23(14):2855-2861 (Jul. 2002).

O'BRIEN, F J, “Biomaterials & scaffolds for tissue engineering,” Mat. Today, 14(3):88-95 (2011).

OLDE DAMINK, L H et al., “Influence of ethylene oxide gas treatment on the in vitro degradation behavior of dermal sheep collagen,”J. Biomed. Mater. Res., 29(2):149-155 (Feb. 1995).

REING, J E et al., “Degradation products of extracellular matrix affect cell migration and proliferation,” Tissue Eng. Part A, 15(3):605-614 (Mar. 2009).

ROVERI, N et al., “Biologically inspired growth of hydroxyapatite nanocrystals inside self-assembled collagen fibers,” Mat. Sci. Eng. C, 23(3):441-446 (Mar. 2003).

RUSSO, L et al., “Carbonate hydroxyapatite functionalization: a comparative study towards (bio)molecules fixation,” Interface Focus, 4(1):20130040 (Feb. 2014).

SCHRODINGER 2013. Schrodinger, LLC: New York, N.Y. (2013).

SINGLETON, P and SAINSBURY, D, “DICTIONARY OF MICROBIOLOGY AND MOLECULAR BIOLOGY,” 2^{nd} Ed., John Wiley and Sons, New York (1987).

TAMPIERI, A et al., “Biologically inspired synthesis of bone-like composite: self-assembled collagen fibers/hydroxyapatite nanocrystals,” J. Biomed. Mater. Res. A, 67(2):618-625 (Nov. 2003).

TAMPIERI, A et al., “Design of graded biomimetic osteochondral composite scaffolds,” Biomaterials, 29(26):3539-3546 (Sept. 2008).

TAMPIERI, A et al., “From biomimetic apatites to biologically inspired composites,” Anal. Bioanal. Chem., 381(3):568-576 (Feb. 2005).

TAMPIERI, A et al., “Mimicking natural bio-mineralization processes: a new tool for osteochondral scaffold development,” Trends Biotechnol., 29(10):526-535 (Oct. 2011).

TAMPIERI, A et al., “Design of graded biomimetic osteochondral composite scaffolds,” Biomaterials, 29(26):3539-3546 (2008).

TARABALLI, F et al., “Amino and carboxyl plasma functionalization of collagen films for tissue engineering applications,” J. Colloid Interface Sci., 394:590-597 (2013).

TATE, M K et al., “Surgical membranes as directional delivery devices to generate tissue: testing in an ovine critical sized defect model,” PloS One, 6(12):e28702 (2011).

TARABALLI, F et al., “Amino and carboxyl plasma functionalization of collagen films for tissue engineering applications,” J. Colloid Interface Sci., 394:590-597 (2013).

TARABALLI, F et al., “Biomimetic collagenous scaffold to tune inflammation by targeting macrophages,” J. Tissue Eng., 2016(7):2041731415624667 (Feb. 2016).

THITISET, T et al., “Development of collagen/demineralized bone powder scaffolds and periosteum-derived cells for bone tissue engineering application,” Int. J. Mol. Sci., 14(1):2056-2071 (Jan. 2013).

UENO, T et al., “Small intestinal submucosa (SIS) in the repair of a cecal wound in unprepared bowel in rats,” J. Gastrointest. Surg., 11(7):918-922 (Jul. 2007).

VALENTIN, J E et al., “Extracellular matrix bioscaffolds for orthopaedic applications. A comparative histologic study,” J. Bone Joint Surg. Am., 88(12):2673-2686 (Dec. 2006).

VASCONCELOS, A et al., “Novel silk fibroin/elastin wound dressings,” Acta Biomaterialia, 8:3049-3060 (2012).

ZANTOP, T et al., “Extracellular matrix scaffolds are repopulated by bone marrow-derived cells in a mouse model of achilles tendon reconstruction,” J. Orthop. Res., 24(6):1299-1309 (Jun. 2006).

ZHANG, C et al., “A study on a tissue-engineered bone using rhBMP-2 induced periosteal cells with a porous nano-hydroxyapatite/collagen/poly (L-lactic acid) scaffold,” Biomed. Mat., 1(2):56 (XXXX 2006).

ZHANG, X et al., “A perspective: engineering periosteum for structural bone graft healing,” Clin. Orthopaed. Rel. Res., 466(8):1777-1787 (2008).

ZHANG, Y N et al., “A highly elastic and rapidly crosslinkable elastin-like polypeptide-based hydrogel for biomedical applications,” Adv. Funct. Mat., 25:4814-4826 (2015).

It should be understood that the examples and embodiments described herein are for illustrative purposes only and that various modifications or changes in light thereof will be suggested to persons skilled in the art and are to be included within the spirit and purview of this application and the scope of the appended claims. All references, including publications, patent applications and patents, cited herein are specifically incorporated herein by reference to the same extent as if each reference was individually and specifically indicated to be incorporated by reference, and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising,” “having,” “including,” or “containing,” with reference to an element or elements is intended to provide support for a similar aspect or embodiment of the invention that “consists of,” “consists essentially of,” or “substantially comprises” the particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of ordinary skill in the art that variations may be applied to the compositions and methods and in the steps or in the sequence of steps of the method described herein without departing from the concept, spirit and scope of the invention. More specifically, it will be apparent that certain agents that are chemically- or physiologically-related may be substituted for the agents described herein while the same or similar results would be achieved. All such similar substitutes and modifications apparent to those ordinarily skilled in the art are deemed to be within the spirit, scope, and concept of the invention as defined by the appended claims.

What is claimed is:

1. A biocompatible, implantable, multilayer delivery system, comprising: (a) at least one elecrospun layer; and (b) at least one porous, nanostructured membrane layer comprising collagen.

2. The biocompatible, implantable, multilayer delivery system of claim 1, wherein the at least one porous, nanostructured membrane layer comprises Type I collagen.

3. The biocompatible, implantable, multilayer delivery system of claim 2, wherein the Type I collagen is obtained from human or bovine tendon.

4. The biocompatible, implantable, multilayer delivery system of claim 1, wherein the at least one electrospun layer is substantially non-porous.

5. The biocompatible, implantable, multilayer delivery system of claim 1, wherein the at least one porous, nanostructured membrane layer further comprises mammalian elastin.

6. The biocompatible, implantable, multilayer delivery system of claim 1, wherein the at least one porous, nanostructured membrane layer further comprises a therapeutic drug or one or more bioactive molecules.

7. The biocompatible, implantable, multilayer delivery system of claim 1, wherein the at least one porous, nanostructured membrane layer further comprises a population of mammalian stem cells, leukosomes, liposomes, nanoparticles, nanovesicles, or combinations thereof.

8. The biocompatible, implantable, multilayer delivery system of claim 1, wherein the at least one porous, nanostructured membrane layer further comprises an analgesic, an anaesthetic, or a combination thereof.

9. The biocompatible, implantable, multilayer delivery system of claim 8, wherein the anaesthetic is benzocaine, bupivacaine, butesin picrate, chloroprocaine, ethyl chloride, fluori-methane, lidocaine, mepivacaine, pramoxine, or a combination thereof.

10. The biocompatible, implantable, multilayer delivery system of claim 8, wherein the analgesic is a COX-2 inhibitor, a non-steroidal anti-inflammatory drug (NSAID), buprenorphine, morphine, or an analog or derivative thereof.

11. The biocompatible, implantable, multilayer delivery system of claim 1, adapted and configured for implantation at a surgical site, a wound, or a structural defect within an organ or a tissue.

12. The biocompatible, implantable, multilayer delivery system of claim 1, adapted and configured for post-surgery implantation, at a site within or about the body of a human.

13. The biocompatible, implantable, multilayer delivery system of claim 1, adapted and configured for surgical implantation within or about a lesion, structural defect or trauma site in a mammalian patient.

14. The biocompatible, implantable, multilayer delivery system of claim 1, further comprising an additional non-porous layer.

15. The biocompatible, implantable, multilayer delivery system of claim 14, wherein the additional non-porous layer provides a structural rigidity suitable for implantation of the delivery system within the heart of a mammalian patient.

16. The biocompatible, implantable, multilayer delivery system of claim 14, wherein the additional non-porous layer permits directional elution of an active agent or a population of cells from within the porous layer.

17. The biocompatible, implantable, multilayer delivery system of claim 1, wherein the interface between layers is substantially seamless.

18. The biocompatible, implantable, multilayer delivery system of claim 17, wherein there is a substantially continuous physical integration between adjacent layers.

19. The biocompatible, implantable, multilayer delivery system of claim 1, wherein the at least one porous, nanostructured membrane layer further comprises a population of mammalian stem cells.

20. The biocompatible, implantable, multilayer delivery system of claim 19, wherein the population of stem cells comprises human bone marrow mesychemal stem cells.

21. The biocompatible, implantable, multilayer delivery system of claim 1, wherein the at least one porous, nanostructured membrane layer comprises a plurality of pores each having an average diameter of between about 1 and about 10 microns.

22. The biocompatible, implantable, multilayer delivery system of claim 1, wherein the porosity of the nanostructured membrane layer is between about 40% and about 80%.

23. The biocompatible, implantable, multilayer delivery system of claim 1, wherein the at least one porous, nanostructured membrane layer further comprises a therapeutic agent selected from the group consisting of analgesics, anaesthetics, angiogenic factors, antibiotics, antibodies, anti-inflammatory agents, anti-pyretics, bioactive peptides, polynucleotides, polypeptides, chemotherapeutics, growth factors, hormones, anti-rejection drugs, and combinations thereof.

24. The biocompatible, implantable, multilayer delivery system of claim 1, wherein the at least one porous, nanostructured membrane layer comprises a medicament selected from the group consisting of growth factors, angiogenic factors, chemotherapeutic agents, anti-rejection agents, and RGD peptides.

25. The biocompatible, implantable, multilayer delivery system of claim 1, wherein crosslinking of the layers alters the porosity or the tortuosity of the delivery system.

26. A therapeutic kit comprising the biocompatible, implantable, multilayer delivery system of claim 1, and instructions for is implantation within a selected tissue site or an organ of a mammalian patient.

27. An implantable device comprising the biocompatible, implantable, multilayer delivery system of claim 1.

28. A bioengineered, electrospun tissue-repair scaffold that comprises the biocompatible, multilayer delivery system of claim 1.

29. An implantable device for promoting wound healing, organ or tissue regeneration or reformation, in a mammal, the device comprising: (a) the biocompatible, implantable, multilayer delivery system of claim 1, and (b) at least one therapeutic agent selected from the group consisting of analgesics, anaesthetics, angiogenic factors, antibiotics, antibodies, anti-inflammatory agents, anti-pyretics, bioactive peptides, polynucleotides, polypeptides, chemotherapeutics, growth factors, hormones, anti-rejection drugs, and combinations thereof.

30. A method for providing localized delivery of a therapeutic agent within one or more organs or tissues of a mammal, the method comprising at least the step of: surgically implanting into a site within the body of a mammalian patient where localized delivery of the therapeutic agent is desired, a biocompatible, multilayer delivery system that comprises: (a) at least one elecrospun layer; (b) at least one porous, nanostructured membrane layer comprising collagen, and (c) at least one therapeutic agent.

31. The method of claim 30, wherein the at least one therapeutic agent is selected from the group consisting of analgesics, anaesthetics, angiogenic factors, antibiotics, antibodies, anti-inflammatory agents, anti-pyretics, bioactive peptides, polynucleotides, polypeptides, chemotherapeutics, growth factors, hormones, anti-rejection drugs, and combinations thereof.

32. The method of claim 30, wherein the at least one porous, nanostructured membrane layer further comprises a plurality of leukosomes, nanoparticles, microparticles, or a combination thereof.

33. The method of claim 31, wherein the at least one therapeutic agent is selected from the group consisting of bupivacaine, novacaine, lidocaine, and combinations thereof, in an amount effective to provide localized analgesia, or the alleviation of post-surgical pain.
